(19) United States
(12) Patent Application Publication
(10) Pub. No.: US 2025/0241923 A1
(43) Pub. Date: Jul. 31, 2025

(54) A DUAL SELECTIVE PI3K DELTA AND GAMMA INHIBITORS AND/OR A SALT-INDUCIBLE KINASE 3 INHIBITOR FOR TREATING SOLID TUMORS

(71) Applicants: Incozen Therapeutics Pvt. Ltd, Hyderabad (IN); Rhizen Pharmaceuticals SA, BASEL (CH)

(72) Inventors: Swaroop Kumar Venkata Satya VAKKALANKA, Basel (CH); Ajit Mohanchandran NAIR, Hyderabad, Telangana (IN); Venkata Satyanarayana ELESWARAPU, Hyderabad (IN)

(21) Appl. No.: 18/845,872

(22) PCT Filed: Apr. 27, 2023
(86) PCT No.: PCT/IB2023/054382

§ 371 Date: Sep. 10, 2024

(30) Foreign Application Priority Data
Apr. 29, 2022 (IN) 202241025286

(51) Int. Cl. A61K 31/52; A61K 31/337; A61K 31/502; A61K 31/5025; A61K 31/565; A61K 31/704; A61K 45/06; A61P 35/00

(52) U.S. Cl. CPC A61K 31/52; A61K 31/337; A61K 31/502; A61K 31/5025; A61K 31/565; A61K 31/704; A61K 45/06; A61P 35/00

(57) ABSTRACT

The present invention relates to the use of a dual selective PI3K delta and gamma (PI3K δ/γ) inhibitor, a Salt-Inducible Kinase 3 (SIK3) inhibitor, or a combination thereof, and pharmaceutical compositions containing them for the treatment of solid tumors, such as breast cancer. The present invention also relates to a compound of formula (A) (also known as Tenalisib, (S)-2-(1-(9H-purin-6-ylamino) propyl)-3-(3-fluorophenyl)-4H-chromen-4-one), a dual selective PI3K delta and gamma (PI3K δ/γ) inhibitor, or a pharmaceutically acceptable salt thereof, or a compound of formula (M) ((S)-3-(3-fluorophenyl)-2-(1-((8-hydroxy-9H-purin-6-yl)amino) propyl)-4H-chromen-4-one), a Salt-Inducible Kinase 3 (SIK3) inhibitor, or a pharmaceutically acceptable salt thereof, or a combination thereof, for the treatment of solid tumors, e.g., breast cancer.

This application is the U.S. national stage of International Application No. PCT/IB2023/054382, filed Apr. 27, 2023, which claims the benefit of Indian Patent Application number 202241025286, filed Apr. 29, 2022, which is hereby incorporated by reference in its entirety.

### FIELD OF THE INVENTION

The present invention relates to the use of a dual selective PI3K delta and gamma (PI3K δ/γ) inhibitor, a Salt-Inducible Kinase 3 (SIK3) inhibitor, or a combination thereof, and pharmaceutical compositions containing them, for the treatment of solid tumors, such as, for example, breast cancer, ovarian cancer, pancreatic cancer, and colon cancer.

### BACKGROUND OF THE INVENTION

Solid tumors are an abnormal mass of tissue that usually does not contain cysts or liquid areas. Solid tumors may be benign (non-cancerous) or malignant (cancerous). Different types of solid tumors are named for the type of cells that form them. Examples of solid tumors include sarcomas and carcinomas. The word tumor does not always imply cancer. In discussing tumors that are malignant (cancerous), different kinds of cancerous solid tumors are named for the type of cells of which they are composed:
Sarcomas—Cancers arising from connective or supporting tissues, such as bone or muscle.
Carcinomas—Cancers arising from the body's glandular cells and epithelial cells present in the skin and the tissue covering or lining the body's organs.

Breast cancer is the most common form of cancer in women and is the second most common cause of cancer-related deaths among women. Breast cancer accounts for 22% of all female cancers and 15% of cancer deaths among women.

Breast cancer is a cancer that develops due to uncontrollable changes in the function or growth of the cells forming breast tissue. These changes transform these cells into cancerous cells that have the ability to spread. Breast cancer can occur in both men and women, but it is more common in women. Risk factors for developing breast cancer include obesity, a lack of physical exercise, alcoholism, hormone replacement therapy during menopause, ionizing radiation, an early age at first menstruation, having children late in life or not at all, older age, having a prior history of breast cancer, and a family history of breast cancer.

Breast cancer can spread when the cancer cells get into the blood or lymph system and are then carried to other parts of the body. If cancer cells have spread to your lymph nodes, there is a higher chance that the cells could have travelled through the lymph system and spread (metastasized) to other parts of your body. Still, not all women with cancer cells in their lymph nodes develop metastases, and some women with no cancer cells in their lymph nodes might develop metastases later. Breast cancer develops because of genetic mutations or damage to DNA. These can be associated with exposure to estrogen, inherited genetic defects, or inherited genes that can cause cancer, such as the BRCA1 and BRCA2 genes.

Types of breast cancer include:
Ductal carcinoma in situ (DCIS), Lobular carcinoma in situ (LCIS) is the most common type of non-invasive breast cancer. It starts in the milk ducts/lobules of the breast and is non-invasive because it hasn't spread into any surrounding breast tissue. DCIS, LCIS isn't life-threatening but having DCIS, LCIS can increase the risk of developing an invasive breast cancer later in life.
Invasive ductal carcinoma (IDC) is the most common type and Invasive lobular carcinoma (ILC) is the second most common type of breast cancer. About 80% of all breast cancers are invasive ductal carcinomas. Invasive ductal carcinoma/Invasive lobular carcinoma means that the cancer that began in the milk ducts/milk producing lobules of the breast has broken through the lining of the milk duct/lobule and spread into the surrounding breast tissue. Over time invasive ductal/lobular breast cancer can spread to the lymph nodes and potentially to other parts of the body.
Paget's disease of the nipple is a rare form of breast cancer in which cancer cells grow in the nipple or the areola (the area around the nipple). The nipple and areola often become scaly, red, itchy, and irritated. Many people with Paget's disease also have either DCIS or invasive breast cancer somewhere else in the breast. The unusual changes in the nipple and areola are often the first signs that breast cancer is present.
Inflammatory breast cancer is a rare and aggressive form of invasive breast cancer that affects the blood vessels in the skin and/or lymphatic vessels of the breast. This causes the breast to become red and inflamed.
Phyllodes tumours of the breast are rare. Although most phyllodes tumours are benign (not cancerous) some are malignant (cancerous). Phyllodes tumours tend to grow quickly, but they rarely spread outside the breast. Phyllodes tumours develop in the breast's connective tissue or stroma (the tissue that holds everything together inside the breast) i.e., outside the ducts and lobules of the breast.
Metastatic breast cancer (MBC) is breast cancer that has spread beyond the breast to other organs in the body, most often the bones, lungs, liver or, less commonly, brain. Metastatic Breast Cancer is often found by a symptom-perhaps a recurring pain or cough, shortness of breath, lack of appetite, headaches, or an injury. It is also possible to diagnose of metastases through routine scans. Metastatic breast cancer is incurable, with a 5-year survival rate of 28%. In the USA, more than 42,000 patients die from MBC every year.

Breast cancer is the most frequent cancer type in women worldwide (Bray et al., CA Cancer J. Clin., 68, 394-424, 2018, doi: 10.3322/caac.21492). Breast cancer is generally divided into three subtypes, with targeted therapies determined by subtypes. Hormone receptor-positive (HR+)/HER2—is the most common subtype and accounts for around 70% of breast cancer patients (Howlader et al., J. Natl. Cancer hist, 106, 2014: dju055). Treatment generally involves endocrine therapy (ET) (Waks et al., J. Am. Med. Assoc., 321, 288-300, 2019). The approval of CDK4/6 inhibitors for use in combination with ET substantially improves survival outcomes (Shah et al., Oncology, 32, 216-222, 2018). However, after resistance develops to hormone therapy, chemotherapy becomes the only standard treatment option (Cardoso et al., J. Natl. Cancer Inst., 101, 1174-1181, 2009, doi. 10.1093/jnci/djp235). 15-20% of patients are diagnosed as HER2+ (Howlader et al., J. Natl. Cancer Inst., 106, 2014,. djuOSS). The standard-of-care treatment for HER2+ breast cancer incorporates HER2-targeted antibody with chemotherapy (Romond et al., N. Engl. J. Med., 353, 1673-1684, 2005; Waks et al.,.J. Am. Med. Assoc., 321, 288-300, 2019). However, even after disease progression upon therapy, continued administration of anti-HER2 and chemotherapy remains as the subsequent treatment (Olson et al., Ann. Oncol., 23, 93-97, 2012, doi. 10.1093/annone/mdr061). Accounting for 10-20% of newly diagnosed breast cancer cases, triple-negative breast cancer (TNBC) is characterized by the lack of hormone receptor expression and lack of HER2/NEU gene overexpression (Dent et al., Clin. Cancer Res, 13, 4429-4434, 2007, doi: 10.1158/1078-0432.ccr-06-3045). TNBC demonstrates high heterogeneity in the mutational profile and shows the highest relapse risks (Dent et al., C′lin. Cancer Res., 13, 4429-4434, 2007, doi. 10.1158/1078-0432.cer-06-3045). Due to a lack of targets, chemotherapy is currently the primary treatment for TNBC, but the clinical benefits are usually not durable due to frequently acquired resistance (Liedtke et al., J. Clin. Oncol, 26, 1275-1281, 2008, doi: 10.1200/jco.2007.14.4147).

Upregulation of the phosphoinositide 3-kinase (PI3K) signalling pathway is commonly observed in breast cancer patients. It has been associated with breast cancer tumorigenesis, progression, and the development of resistance to hormone therapy and chemotherapy (Guerrero-Zotano et al., Cancer Metastasis Rev., 35, 515-524, 2016, doi: 10.1007/s10555-016-9637-x; Drullinsky et al., Breast Cancer Res. Treatment. 181, 233-248 (2020). doi: 10.1007/310549-020-05618-1). Therefore, it is essential to elucidate the mechanism of the PI3K signalling pathway in breast cancer and explore the potential of PI3K inhibitors in the treatment of different subtypes of breast cancer, alone or combined with other therapies in both early and metastatic settings.

Pathological activation of the PI3K pathway is among the most frequent signalling events associated with cellular transformation, cancer formation and metastasis in solid tumors (Okkenhaug et. al., Cancer Discovery, 6 (10), 1090-1105, 2016). This is exemplified by the frequent activating mutations in PIK3CA and the loss of PTEN (Phosphatase and Tensin Homolog) functionality in common cancers such as breast, colon and ovary. PIK3CD is mostly non-mutated in cancer, however the expression levels of p1108 protein seem to act as an intrinsic cancer-causing driver in various solid tumors including breast, prostate, colorectal, liver, merkel-cell carcinoma, glioblastoma, and neuroblastoma. (Lydia Xenou et. al., Clin. Sci. (Lond.), 134 (12), 1377-1397, 2020). Recent studies have revealed that the oncogenic potential of overexpressed p1108 PI3K correlate with suppressed PTEN lipid phosphatase activity in cancer cells. Given that PTEN somatic mutations occur in a small percentage of human breast cancers and that p1108 is highly expressed in this tumor type, PI3K8 inhibitors may provide an opportunity in the intervention of breast cancer (Tzenaki et. al., The FASEB Journal, 26 (6), 2498-2508, 2012). Recently, p1108 selective inhibitors are being studied as potential single agent treatment or in combination to improve responses in patients with solid tumors.

The immunosuppressive microenvironment in the tumor tissue is partly mediated by non-tumoral stromal cells, most notably tumor associated macrophages (TAMs) (Ge Z, Ding S., Front Oncol., 10, 590941, 2020). Data has shown that PI3Ky acts as a molecular switch turning on immunosuppression while shutting down immune stimulatory activities. Inhibitors of PI3Ky thus can cause a shift/re-programming leading to enhanced adaptive immunity, including increased recruitment and cytotoxicity of T cells. This change in the immune environment significantly reduces the growth and metastasis of tumors (Kaneda, et. al., Nature, 539 (7629), 437-442, 2016).

Salt-Inducible kinases (SIKs) are highly conserved serine/threonine protein kinases that belong to a family of AMP-activated protein kinases (AMPKs) and have a role in steroidogenesis, adipogenesis and regulation of tumors. SIKs are dysregulated in various cancers, including ovarian, breast, prostate, and lung cancers, indicating that SIKs may have role in tumor occurrence or progression. SIK3 facilitates tumor cell resistance against cytotoxic T cell attack by shifting TNF signalling from apoptosis to survival. SIK3-specific inhibitors alone sensitize a wide array of human and murine cancer cell lines to TNF-induced apoptosis by engaging the HDAC4/NFkB pathway. Thus, targeting SIK3 to re-sensitize tumors to immune attack is a compelling therapeutic strategy for cancer treatment.

SIK3 is highly expressed in around 55% of breast cancer patients, and markedly governs G1/S process through upregulating the gene expression of cyclin D and cyclin E, simultaneously downregulating the expression of p21 and p27, or increasing the cyclin dependent kinase 2 (CDK2) activity. The absence of SIK3 leads to prolongation of mitosis in mice and human cells, thus increasing the sensitivity of cancer cells to a variety of anti-mitotic drugs. Studies have also demonstrated that SIK3 expression is upregulated in breast cancer promoting aerobic glycolysis and tumor growth by activating mTOR/Akt signalling pathway. SIK3 also plays a positive role in mediating the high salt-induced inflammatory signal response that leads to cancer cell proliferation. SIK3 induces upregulation of inflammatory arginine. metabolism factors, such as iNOS and ass-1, and the downregulation of anti-inflammatory enzymes, such as arginase-1 and ornithine decarboxylase in breast cancer.

Despite some progress made in treatment in breast cancer, challenges remain in the treatment, side effects and desired clinical benefits of patients. Accordingly, there still remains an unmet need for drugs for the treatment of solid tumours, such as, for example, breast cancer.

### SUMMARY OF THE INVENTION

The present inventors have surprisingly found that a dual selective PI3K delta and gamma (PI3K δ/γ) inhibitor, a Salt-Inducible Kinase 3 (SIK3) inhibitor, or a combination thereof, optionally in combination with other drugs, show excellent activity against solid tumours, such as, for example, pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, and soft tissue sarcoma, more importantly, colon cancer, ovarian cancer, pancreatic cancer, and breast cancers, such as, for example, metastatic breast cancer, hormone receptor (HR) positive and HER2 negative breast cancer and triple negative breast cancer (TNBC).

Accordingly, in one aspect, the present invention relates to (i) a compound of formula (A) (also known as Tenalisib, (S)-2-(1-(9H-purin-6-ylamino) propyl)-3-(3-fluorophenyl)-4H-chromen-4-one), a dual selective PI3K delta and gamma (PI3K δ/γ) inhibitor, or a pharmaceutically acceptable salt thereof, (ii) a compound of formula (M) ((S)-3-(3-fluorophenyl)-2-(1-((8-hydroxy-9H-purin-6-yl)amino) propyl)-4H-chromen-4-one), a Salt-Inducible Kinase 3 (SIK3) inhibitor, or a pharmaceutically acceptable salt thereof, or (iii) a combination thereof for use in the treatment of a solid tumor, such as, e.g., breast cancer, colon cancer, ovarian cancer, and pancreatic cancer.

Another embodiment is a pharmaceutical composition comprising (i) a dual selective PI3K delta and gamma (PI3K δ/γ) inhibitor and (ii) a Salt-Inducible Kinase 3 (SIK3) inhibitor.

Yet another embodiment is a pharmaceutical composition comprising (i) a compound of formula (A) or a pharmaceutically acceptable salt thereof, (ii) a compound of formula (M) or a pharmaceutically acceptable salt thereof, or (iii) a combination thereof for use in the treatment of a solid tumor, such as, e.g., breast cancer, colon cancer, ovarian cancer, and pancreatic cancer.

The compound of formula (M) is a major metabolite of the compound of formula (A) in humans.

The structures of the compound of formula (A) and the compound of formula (M) are shown below.

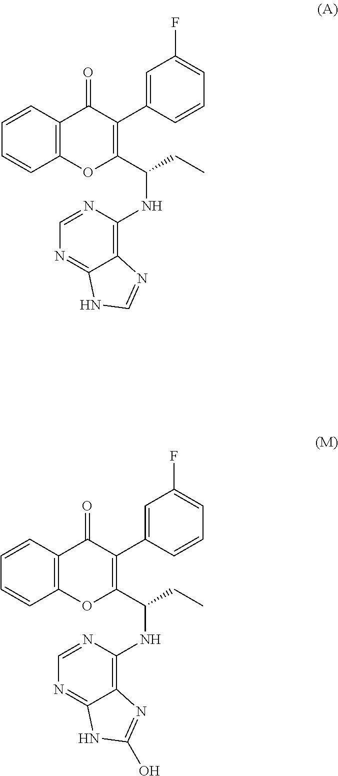 [SMILES: CC[C@H](Nc1ncnc2[nH]cnc12)c1oc2ccccc2c(=O)c1-c1cccc(F)c1 ; CC[C@H](Nc1ncnc2[nH]c(O)nc12)c1oc2ccccc2c(=O)c1-c1cccc(F)c1]

The compound of formula (A) (also referred to herein as Compound A) and its preparation is disclosed in International Publication No. WO 14/195888, which is hereby incorporated by reference in its entirety. The compound of formula (M) (also referred to herein as Compound M) and its preparation is disclosed in International Publication No. WO 21/229452, which is hereby incorporated by reference in its entirety.

In another aspect, the present invention relates to a method of treating solid tumors (such as breast cancer) comprising administering to a subject (e.g., a human subject in need thereof) a dual selective PI3K delta and gamma (PI3K δ/γ) inhibitor, a Salt-Inducible Kinase 3 (SIK3) inhibitor, or a combination thereof.

Yet another aspect is the use of a dual selective PI3K delta and gamma (PI3K δ/γ) inhibitor, a Salt-Inducible Kinase 3 (SIK3) inhibitor, or a combination thereof for the treatment of solid tumors (such as breast cancer, ovarian cancer, pancreatic cancer, or colon cancer) in a subject (such as a human subject in need thereof).

In certain embodiments of any of the methods and uses described herein, the solid tumor is selected from carcinomas, sarcomas, or any combination thereof.

In certain embodiment of any of the methods and uses described herein, the solid tumor is selected from pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, and soft tissue sarcoma.

In certain embodiments of any of the methods and uses described herein, the solid tumor is selected from non-small cell lung cancer, small-cell lung cancer, colon cancer, CNS cancer, melanoma, ovarian cancer, renal cancer, prostate cancer, and breast cancer.

In certain embodiments of any of the methods and uses described herein, the solid tumor is selected from breast cancer, colon cancer, ovarian cancer, and pancreatic cancer.

In certain embodiments of any of the methods and uses described herein, the solid tumor is ovarian cancer.

In certain embodiments of any of the methods and uses described herein, the solid tumor is pancreatic cancer.

In certain embodiments of any of the methods and uses described herein, the solid tumor is colon cancer.

In certain embodiments of any of the methods and uses described herein, the solid tumor is breast cancer.

In certain embodiments of any of the methods and uses described herein, the breast cancer is selected from invasive breast cancer, non-invasive breast cancer, cancerous phyllodes tumors of the breast, locally advanced or metastatic breast cancer, hormone receptor (HR) positive and HER2 negative breast cancer, hormone receptor (HR) positive and HER2 positive breast cancer, hormone receptor (HR) negative and HER2 positive breast cancer, hormone receptor (HR) negative and HER2 negative breast cancer (triple negative breast cancer-TNBC).

In certain embodiments of any of the methods and uses described herein, the breast cancer is selected from invasive breast cancer, non-invasive breast cancer, and cancerous phyllodes tumors of the breast.

In certain embodiments of any of the methods and uses described herein, the breast cancer is selected from locally advanced or metastatic breast cancer.

In certain embodiments of any of the methods and uses described herein, the breast cancer is selected from hormone receptor (HR) positive and HER2 negative breast cancer, hormone receptor (HR) positive and HER2 positive breast cancer, hormone receptor (HR) negative and HER2 positive breast cancer, and hormone receptor (HR) negative and HER2 negative breast cancer (triple negative breast cancer-TNBC).

In certain embodiments of any of the methods and uses described herein, the breast cancer is hormone receptor (HR) positive and HER2 negative breast cancer.

In certain embodiments of any of the methods and uses described herein, the breast cancer is triple negative breast cancer.

In certain embodiments of any of the methods and uses described herein, the breast cancer is selected from metastatic breast cancer, hormone receptor (HR) positive and HER2 negative breast cancer, and triple negative breast cancer (TNBC).

In another aspect, the present invention is directed to a method of treating solid cancers (e.g., breast cancer, ovarian cancer, pancreatic cancer, colon cancer) comprising administering to a subject (e.g., a human subject in need thereof) a dual selective PI3K delta and gamma (PI3K δ/γ) inhibitor, a pharmaceutical composition comprising a dual selective PI3K delta and gamma (PI3K δ/γ) inhibitor.

In certain embodiments of any of the methods and uses described herein, the dual selective PI3K delta and gamma inhibitor is a compound of formula (A), or a pharmaceutically acceptable salt thereof.

Another aspect of the present invention is the use of a compound of formula (A), or a pharmaceutically acceptable salt thereof, a compound of formula (M), or a pharmaceutically acceptable salt thereof, or a combination thereof, for the preparation of a medicament for the treatment of solid tumours.

In certain embodiments of any of the methods and uses described herein, the subject is a human.

In certain embodiments of any of the methods and uses described herein, the compound of formula (A), or a pharmaceutically acceptable salt thereof, the compound of formula (M), or a pharmaceutically acceptable salt thereof, or the combination thereof, is administered by the oral route, the intravenous route, the intramuscular route, or the intraperitoneal route. In humans, a conventional administration route is the oral route.

In certain embodiments of any of the methods and uses described herein, the compound of formula (A), or a pharmaceutically acceptable salt thereof, the compound of formula (M), or a pharmaceutically acceptable salt thereof, or the combination thereof, is used for the preparation of a medicament for the treatment of breast cancer that is administered by the oral route.

Yet another aspect of the present invention is a method of inhibiting dual PI3K delta and gamma kinase in a breast cancer patient comprising administering to the patient an effective amount of a compound of formula (A), or a pharmaceutically acceptable salt thereof, a compound of formula (M), or a pharmaceutically acceptable salt thereof, or a combination thereof.

In another aspect, the present invention also provides a method of treating solid tumours in a subject (e.g., a human subject in need thereof) comprising administering to the subject a compound of formula (A), or a pharmaceutically acceptable salt thereof, a compound of formula (M), or a pharmaceutically acceptable salt thereof, or a combination thereof.

In another aspect, the present invention relates to a pharmaceutical composition for the treatment of solid tumours comprising a compound of formula (A), or a pharmaceutically acceptable salt thereof, and a pharmaceutically acceptable carrier.

In another aspect, the present invention relates to a pharmaceutical composition for the treatment of solid tumours comprising a compound of formula (M), or a pharmaceutically acceptable salt thereof, and a pharmaceutically acceptable carrier.

In certain embodiments of any of the methods and uses described herein, the compound of formula (A), or a pharmaceutically acceptable salt thereof, is administered for the first-line treatment of breast cancer and/or the treatment of non-resectable breast cancer.

In certain embodiments of any of the methods and uses described herein, the compound of formula (A), or a pharmaceutically acceptable salt thereof, is administered for the treatment of a human subject suffering from relapsed breast cancer, refractory breast cancer, or relapsed-refractory breast cancer.

In certain embodiments of any of the methods and uses described herein, the dual selective PI3K delta and gamma (PI3K δ/γ) inhibitor and/or Salt-Inducible Kinase 3 (SIK3) inhibitor is administered orally.

In certain embodiments of any of the methods and uses described herein, the compound of formula (A), or a pharmaceutically acceptable salt thereof, is administered orally.

In certain embodiments of any of the methods and uses described herein, the compound of formula (A), or a pharmaceutically acceptable salt thereof, is administered (e.g., orally) at a dosage range of about 25 to about 2400 mg per day, such as about 50 to about 2000 mg per day, about 100 to about 1600 mg per day, about 200 to about 1200 mg per day, or about 400 to about 800 mg per day.

In certain embodiments of any of the methods and uses described herein, the compound of formula (A), or a pharmaceutically acceptable salt thereof, is administered (e.g., orally) at a dosage range of about 200 to about 1200 mg twice daily (i.e., a total of about 400 to 2400 mg per day), or about 400 to about 800 mg twice daily (i.e., a total of about 800 to 1600 mg per day).

In certain embodiments of any of the methods and uses described herein, the compound of formula (A), or a pharmaceutically acceptable salt thereof, is administered (e.g., orally) as single or in divided doses (e.g., two, three or four doses per day).

In additional embodiments of any of the methods and uses described herein, the compound of formula (A), or a pharmaceutically acceptable salt thereof, is used in combination (administered together or sequentially) with an anti-cancer treatment, an endocrine therapy, one or more cytostatic, cytotoxic or anticancer agents, targeted therapy, or any combination of any of the foregoing.

In one embodiment of any of the pharmaceutical compositions described herein, the pharmaceutical composition further comprises one or more cytostatic, cytotoxic and/or anticancer agents.

In another aspect, the present invention relates to a method of treating solid cancers (e.g., breast cancer) comprising administering to a subject (e.g., a human subject in need thereof) a Salt-Inducible Kinase 3 (SIK3) inhibitor.

In additional embodiments of any of the methods and uses described herein, the SIK-3 inhibitor is a compound of formula (M) or a pharmaceutically acceptable salt thereof.

In another aspect, the present invention relates to the use of a compound of formula (M) or a pharmaceutically acceptable salt thereof, for the preparation of a medicament for the treatment of breast cancer.

In certain embodiments of any of the methods and uses described herein, the subject is a human.

In certain embodiments of any of the methods and uses described herein, the compound of formula (M) or a pharmaceutically acceptable salt thereof, is administered by the oral route, the intravenous route, the intramuscular route, or the intraperitoneal route.

Another aspect of the present invention is the use of the compound of formula (M), or a pharmaceutically acceptable salt thereof, is used for the preparation of a medicament for the treatment of breast cancer that is administered by the oral route.

Yet another aspect of the present invention is a method of inhibiting a Salt-Inducible Kinase 3 (SIK3) in a breast cancer patient (e.g., breast cancer human patient) comprising administering to the patient an effective amount of the compound of formula (M) or a pharmaceutically acceptable salt thereof.

The present invention also provides a method of treating breast cancer in a subject (e.g., a human subject in need thereof) comprising administering to the subject a compound of formula (M), or a pharmaceutically acceptable salt thereof.

In another aspect, the present invention relates to a pharmaceutical composition for the treatment of breast cancer comprising a compound of formula (M), or pharmaceutically acceptable salt thereof, and a pharmaceutically acceptable carrier.

In additional embodiments of any of the methods and uses described herein, the compound of formula (M) or a pharmaceutically acceptable salt thereof, is administered for the first line treatment of breast cancer and/or the treatment of non-resectable breast cancer.

In additional embodiments of any of the methods and uses described herein, the compound of formula (M) or a pharmaceutically acceptable salt thereof, is administered (e.g., orally) for the treatment of human subject suffering from relapsed breast cancer, refractory breast cancer, or relapsed-refractory breast cancer.

In additional embodiments of any of the methods and uses described herein, the compound of formula (M) or a pharmaceutically acceptable salt thereof, is administered orally.

In additional embodiments of any of the methods and uses described herein, the compound of formula (M) or a pharmaceutically acceptable salt thereof, is administered (e.g., orally) at a dosage range of about 25 to about 2400 mg per day, such as about 50 to about 2000 mg per day, about 100 to about 1600 mg per day, about 200 to about 1200 mg per day, or about 400 to about 800 mg per day.

In additional embodiments of any of the methods and uses described herein, the compound of formula (M) or a pharmaceutically acceptable salt thereof, is administered (e.g., orally) at a dosage range of about 200 to about 1200 mg twice daily (i.e., a total of about 400 to 2400 mg per day), or about 400 to about 800 mg twice daily (i.e., a total of about 800 to 1600 mg per day).

In additional embodiments of any of the methods and uses described herein, the compound of formula (M) or a pharmaceutically acceptable salt thereof, is administered as single or in divided doses (e.g., two, three of four doses daily).

In additional embodiments of any of the methods and uses described herein, the compound of formula (M) or a pharmaceutically acceptable salt thereof, is used in combination (administered together or sequentially) with an anti-cancer treatment, an endocrine therapy, one or more cytostatic, cytotoxic or anticancer agents, targeted therapy, or any combination of any of the foregoing.

In one embodiment of any of the pharmaceutical compositions described herein, the pharmaceutical composition further comprises one or more cytostatic, cytotoxic and/or anticancer agents, or any combination of any of the foregoing.

Another aspect of the present invention is a method of inhibiting SIK3 in a patient with breast cancer comprising administering to the patient an effective amount of a SIK3 compound according to any of the embodiments described herein.

In another aspect, the present invention relates to a method of treating solid cancers (e.g., breast cancer) in a subject (e.g., a subject in need thereof) comprising administering to the subject a dual selective PI3K delta and gamma (PI3K δ/γ) inhibitor and a Salt-Inducible Kinase 3 (SIK3) inhibitor.

In one embodiment of any of the methods and uses described herein, the dual selective PI3K delta and gamma inhibitor is a compound of formula (A) or a pharmaceutically acceptable salt thereof, and the SIK-3 inhibitor is a compound of formula (M) or a pharmaceutically acceptable salt thereof.

In another aspect, the present invention relates to the use of a compound of formula (A) or a pharmaceutically acceptable salt thereof, in combination with a compound of formula (M) or a pharmaceutically acceptable salt thereof, for the preparation of a medicament for the treatment of breast cancer.

In one embodiment of any of the methods and uses described herein, a compound of formula (A) or a pharmaceutically acceptable salt thereof, and a compound of formula (M) or a pharmaceutically acceptable salt thereof, are administered by the oral route, the intravenous route, the intramuscular route, or the intraperitoneal route.

In one embodiment of any of the methods and uses described herein, a compound of formula (A) or a pharmaceutically acceptable salt thereof, and a compound of formula (M) or a pharmaceutically acceptable salt thereof, are administered by the oral route.

In another aspect, the present invention relates to the use of a compound of formula (A) or a pharmaceutically acceptable salt thereof, in combination with a compound of formula (M) or a pharmaceutically acceptable salt thereof, for the preparation of a medicament for the treatment of breast cancer, wherein the compound of formula (A) or a pharmaceutically acceptable salt thereof, and the compound of formula (M) or a pharmaceutically acceptable salt thereof, are administered by the oral route.

The present invention also relates to a method of treating breast cancer in a subject (e.g., a subject in need thereof) comprising administering a dual selective PI3K delta and gamma inhibitor compound of formula (A) or a pharmaceutically acceptable salt thereof, in combination with a SIK3 inhibitor compound of formula (M) or a pharmaceutically acceptable salt thereof.

The present invention further relates to a pharmaceutical composition (e.g., a pharmaceutical composition for the treatment of a solid tumor such as breast cancer comprising a compound of formula (A) or a pharmaceutically acceptable salt thereof, in combination with a compound of formula (M) or a pharmaceutically acceptable salt thereof, and a pharmaceutically acceptable carrier.

In additional embodiments of any of the methods and uses described herein, a combination of a compound of formula (A) or a pharmaceutically acceptable salt thereof, and a compound of formula (M) or a pharmaceutically acceptable salt thereof, are administered for the first line treatment of breast cancer and/or the treatment of non resectable breast cancer.

In additional embodiments of any of the methods and uses described herein, a combination of a compound of formula (A) or a pharmaceutically acceptable salt thereof, and a compound of formula (M) or a pharmaceutically acceptable salt thereof, are administered for the treatment of human subject suffering from relapsed breast cancer, refractory breast cancer, and/or relapsed-refractory breast cancer.

In additional embodiments of any of the methods and uses described herein, a combination of a compound of formula (A) or a pharmaceutically acceptable salt thereof, and a compound of formula (M) or a pharmaceutically acceptable salt thereof, are administered orally.

In additional embodiments of any of the methods and uses described herein, a combination of a compound of formula (A) or a pharmaceutically acceptable salt thereof, and a compound of formula (M) or a pharmaceutically acceptable salt thereof, is administered (e.g., orally) at a dosage range of about 25 to about 2400 mg per day, such as about 50 to about 2000 mg per day, about 100 to about 1600 mg per day, about 200 to about 1200 mg per day, or about 400 to about 800 mg per day.

In additional embodiments of any of the methods and uses described herein, a combination of a compound of formula (A) or a pharmaceutically acceptable salt thereof, and a compound of formula (M) or a pharmaceutically acceptable salt thereof, is administered (e.g., orally) to the subject at a dose of about 25 mg to about 2400 mg, about 25 mg to 2 about 2000 mg, about 25 mg to about 1800 mg, about 25 mg to about 1600 mg, about 25 mg to about 1200 mg, about 25 mg to about 1000 mg, about 25 mg to about 800 mg, about 25 mg to about 600 mg, about 25 mg to about 400 mg, or about 25 mg to 200 mg.

In additional embodiments of any of the methods and uses described herein, a combination of a compound of formula (A) or a pharmaceutically acceptable salt thereof, and a compound of formula (M) or a pharmaceutically acceptable salt thereof, is administered (e.g., orally) to the subject at a dose of about 100 mg to about 2400 mg, about 100 mg to about 2000 mg, about 100 mg to about 1800 mg, about 100 mg to about 1600 mg, about 100 mg to about 1200 mg, about 100 mg to about 1000 mg, about 100 mg to about 800 mg, about 100 mg to about 600 mg, about 100 mg to about 400 mg, or about 100 mg to about 200 mg.

In additional embodiments of any of the methods and uses described herein, a combination of a compound of formula (A) or a pharmaceutically acceptable salt thereof, and a compound of formula (M) or a pharmaceutically acceptable salt thereof, is administered (e.g., orally) at a dosage range of about 200 to about 1200 mg twice daily (i.e., a total of about 400 to 2400 mg per day), or about 400 to about 800 mg twice daily (i.e., a total of about 800 to 1600 mg per day).

In additional embodiments of any of the methods and uses described herein, a combination of a compound of formula (A) or a pharmaceutically acceptable salt thereof, and a compound of formula (M) or a pharmaceutically acceptable salt thereof, are each administered independently as single or in divided doses.

In yet a further aspect, the present invention relates to a method of treating breast cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with an anti-cancer treatment, an endocrine therapy, one or more cytostatic, cytotoxic or anticancer agents, targeted therapy, or any combination or any of the foregoing.

In yet aspect, the present invention relates to method of treating breast cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more cytostatic, cytotoxic or anticancer agents, or any combination or any of the foregoing.

In yet another aspect, the present invention relates to a method of treating breast cancer in a subject (e.g., a subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anti-cancer treatments, one or more cytostatic, cytotoxic or anticancer agents, targeted therapy, or any combination or any of the foregoing.

In one aspect, the present invention relates to a method of treating a solid cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anticancer agents, one or more cytostatic or cytotoxic agents.

In another aspect, the present invention relates to a method of treating a solid cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anticancer agents, one or more cytostatic or cytotoxic agents, or any combination of any of the foregoing, such as, e.g., one or more PARP inhibitors, CDK inhibitors, MEK inhibitors, B-RAF inhibitors, m-TOR inhibitors, selective estrogen receptor degrader (SERD), HER-2 or EGFR inhibitors or dual inhibitors, PD1 inhibitors, RET inhibitors, or any combination of any of the foregoing.

In one aspect, the present invention relates to a method of treating solid cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anticancer agents, one or more cytostatic or cytotoxic agents, or any combination of any of the foregoing, such as, e.g., one or more PARP inhibitors, e.g., olaparib, rucaparib, niraparib, talozoparib, or any combination thereof.

In one aspect, the present invention relates to a method of treating solid cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more cytostatic or cytotoxic agents, such as one or more selective estrogen receptor degrader (SERD), e.g., fulvestrant, brilanestrant, elacestrant, or any combination of any of the foregoing.

In one aspect, the present invention relates to a method of treating breast cancer in a subject (e.g., a subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anti-cancer treatments.

In one embodiment, the present invention relates to a method of treating breast cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anti-cancer treatments such as chemotherapy, radiation therapy, biological therapy, bone marrow transplantation, or stem cell transplantation, alone or in any combination of any of the foregoing.

In one embodiment, the present invention relates to a method of treating breast cancer in a subject (e.g., a human subject in need thereof) comprising administering to the subject a combination of a compound of formula (A) or a pharmaceutically acceptable salt thereof, and a compound of formula (M) or a pharmaceutically acceptable salt thereof, in combination (administered together or sequentially) with one or more anti-cancer treatments such as chemotherapy agents, selected from, e.g., taxol, doxorubicin, daunorubicin, doxorubicin liposomal, epirubicin, idarubicin, valrubicin, carboplatin, carmustine, cisplatin, cyclophosphamide, dacarbazine, ifosfamide, lomustine, melphalan, temozolomide, trabectedin, 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, trifluridine, tipiracil, and any combination of any of the foregoing.

In yet another aspect, the present invention relates to a method of inhibiting PI3K delta and gamma and SIK3 in a subject (e.g., a human subject in need thereof) comprising administering to the subject an effective amount of a dual selective PI3K delta and gamma (PI3K δ/γ) inhibitor (e.g., a compound of formula (A) or a pharmaceutically acceptable salt thereof), in combination (individually or together) with a Salt-Inducible Kinase 3 (SIK3) inhibitor (e.g., a compound of formula (M) or a pharmaceutically acceptable salt thereof). In one embodiment, the subject suffers from a solid tumor. In another embodiment, the administration is oral administration.

In yet another aspect, the present invention relates to a method of inhibiting PI3K, in particular PI3K delta and gamma kinase, and SIK3 in a subject (e.g. a subject in need thereof) comprising administering to the subject an effective amount of a dual selective PI3K delta and gamma (PI3K δ/γ) inhibitor (e.g., a compound of formula (A), or a pharmaceutically acceptable salt thereof), in combination (individually or together) with a Salt-Inducible Kinase 3 (SIK3) inhibitor (e.g., a compound of formula (M), or a pharmaceutically acceptable salt thereof). In one embodiment, the subject suffers from a solid tumor. In another embodiment, the administration is oral administration.

In yet another aspect, the present invention relates to a pharmaceutical composition for the treatment of breast cancer comprising a compound of formula (A) or a pharmaceutically acceptable salt thereof, and a compound of formula (M) or a pharmaceutically acceptable salt thereof.

### BRIEF DESCRIPTION OF THE FIGURES

FIGS. 1A and 1B show the inhibition of cell growth in ZR. 75.1 breast cancer cell lines in the absence of B-estradiol due to (i) fulvestrant (5 or 10 μM), (ii) a combination of Compound A and Compound M (3, 5, or 10 μM), and (iii) a combination of fulvestrant (5 μM or 10 μM) in combination with Compound A and Compound M (3, 5, or 10 μM).

FIG. 1C shows the cell cycle (including G0/G1 cell cycle arrest) in ZR.75.1 cells in the absence of B-estradiol when treated with (i) DMSO, (ii) a combination of Compound A and Compound M (3 or 10 μM), (iii) fulvestrant (3 or 10 μM), and (iv) a combination of fulvestrant (3 μM or 10 μM) in combination with Compound A and Compound M (3 or 10 μM).

FIGS. 2A and 2B show the inhibition of cell growth in ZR. 75.1 breast cancer cell lines in the presence of estrogen due to (i) fulvestrant (3 or 10 μM), (ii) a combination of Compound A and Compound M (3, 5, or 10 μM), and (iii) a combination of fulvestrant (3 or 10 μM) in combination with Compound A and Compound M (3, 5, or 10 μM).

FIG. 2C shows the cell cycle (including G0/G1 cell cycle arrest) in ZR.75.1 cells in the presence of B-estradiol when treated with (i) DMSO, (ii) a combination of Compound A and Compound M (3 or 5 M), (iii) fulvestrant (1 or 3 μM), and (iv) a combination of fulvestrant (1 μM or 3 μM) in combination with Compound A and Compound M (3 or 5 M).

FIGS. 3A and 3B show the inhibition of cell growth in MCF-7 breast cancer cell lines in the presence of B-estradiol due to (i) fulvestrant (3 or 5 μM), (ii) a combination of Compound A and Compound M (3, 5, or 10 μM), and (iii) a combination of fulvestrant (3 μM) in combination with Compound A and Compound M (3, 5, or 10 μM).

FIGS. 4A and 4C show the inhibition of cell growth in MCF-7 and MDA-MB-231 breast cancer cell lines due to (i) Compound M (5 μM), (ii) Compound A (5 μM), (iii) taxol (3 nM), and (iv) the combination of Compound M (5 μM), Compound A (5 μM), and taxol (3 nM).

FIG. 4B shows the inhibition of cell growth in MCF-7 breast cancer cell lines due to (i) Compound M (5 μM), (ii) Compound A (5 μM), (iii) doxorubicin (50 nM), and (iv) the combination of Compound M (5 μM), Compound A (5 M), and doxorubicin (50 nM).

FIG. 5 shows the inhibition of cell growth in MCF-7 breast cancer cell lines due to (i) Compound M (1 μM), (ii) olaparib (5 M), and (iii) the combination of Compound M (1 μM) and olaparib (5 μM).

FIG. 6A shows the inhibition of cell growth in OVCAR-3 ovarian cancer cell lines due to (i) Compound M (5 μM), (ii) talazoparib (0.1 μM), and (iii) the combination of Compound M (5 μM) and talazoparib (0.1 μM).

FIG. 6B shows the inhibition of cell growth in SK-OV-3 ovarian cancer cell lines due to (i) Compound M (5 μM), (ii) talazoparib (3 μM), and (iii) the combination of Compound M (5 μM) and talazoparib (3 μM).

FIG. 6C shows the inhibition of cell growth in OVCAR-3 ovarian cancer cell lines due to (i) Compound M (5 μM), (ii) olaparib (3 μM), and (iii) the combination of Compound M (5 μM) and olaparib (3 μM).

FIG. 7 shows the inhibition of cell growth in SK-OV-3 ovarian cancer cell lines due to (i) DMSO, (ii) Compound M (3 μM), (ii) taxol (6 nM), and (iii) the combination of Compound M (3 μM) and taxol (6 nM).

FIGS. 8A and 8B show the inhibition of cell growth in PANC-1 and MIA paca-2 pancreatic cancer cell lines due to 1, 3, or 10 μM of a combination of a Compound of formula (M) and a compound of formula (A) at a 1:1 ratio (1 μM).

FIG. 9 shows tumor volume distribution on Day 22 in a MC38 syngeneic murine colon carcinoma C57BL/6 model treated with (i) a vehicle, (ii) an anti-PD-1 antibody (clone RMP1-14), (iii) tenalisib, or (iv) a combination of the anti-PD-1 RMP1-14 antibody and tenalisib.

### DETAIL DESCRIPTION OF THE INVENTION

Unless defined otherwise, all technical and scientific terms used herein have the same meaning as is commonly understood in the field to which the subject matter belongs. In the event that there is a plurality of definitions for terms herein, those in this section prevail. Where reference is made to a URL or other such identifier or address, it is understood that such identifiers generally change and particular information on the internet comes and goes, but equivalent information is found by searching the internet. Reference thereto evidences the availability and public dissemination of such information.

It is to be understood that the foregoing general description and the following detailed description are exemplary and explanatory only and are not restrictive of any subject matter. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definitions of standard chemistry and molecular biology terms may be found in reference works, including, but not limited to, Carey and Sundberg “ADVANCED ORGANIC CHEMISTRY 4^{th} edition” Vols. A (2000) and B (2001), Plenum Press, New York and “MOLECULAR BIOLOGY OF THE CELL 5^{th} edition” (2007), Garland Science, New York. Unless otherwise indicated, conventional methods of mass spectroscopy, nuclear magnetic resonance (NMR), high pressure liquid chromatography (HPLC), protein chemistry, biochemistry, recombinant DNA techniques and pharmacology are contemplated within the scope of the embodiments disclosed herein.

Unless specific definitions are provided, the nomenclature employed in connection with, and the laboratory procedures and techniques of, analytical chemistry, and medicinal and pharmaceutical chemistry described herein are those generally used. In some embodiments, standard techniques are used for chemical analyses, pharmaceutical preparation, formulation, and delivery, and treatment of patients. In other embodiments, standard techniques are used for recombinant DNA, oligonucleotide synthesis, and tissue culture and transformation (e.g., electroporation, lipofection). In certain embodiments, reactions and purification techniques are performed e.g., using kits of manufacturer's specifications or as described herein. The foregoing techniques and procedures are generally performed of conventional methods and as described in various general and more specific references that are cited and discussed throughout the present specification.

The term “acceptable” with respect to a formulation, composition or ingredient, as used herein, means having no persistent detrimental effect on the general health of the subject being treated.

The term “pharmaceutically acceptable,” as used herein, refers a material, such as a carrier or diluent or a salt of a compound, which does not abrogate the biological activity or properties of the compound, and is relatively nontoxic, i.e., the material is administered to an individual without causing undesirable biological effects or interacting in a deleterious manner with any of the components of the composition in which it is contained.

Pharmaceutically acceptable salts forming part of this invention include, but are not limited to, salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, thiamine, and the like; chiral bases like alkylphenylamine, glycinol, and phenyl glycinol, salts of natural amino acids such as glycine, alanine, valine, leucine, isoleucine, norleucine, tyrosine, cystine, cysteine, methionine, proline, hydroxy proline, histidine, omithine, lysine, arginine, and serine; quaternary ammonium salts of the compounds of invention with alkyl halides, and alkyl sulphates such as Mel and (Me)₂SO₄, non-natural amino acids such as D-isomers or substituted amino acids; guanidine, substituted guanidine wherein the substituents are selected from nitro, amino, alkyl, alkenyl, alkynyl, ammonium or substituted ammonium salts and aluminum salts. Salts may include acid addition salts where appropriate which are, sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates. Pharmaceutically acceptable solvates may be hydrates or comprise other solvents of crystallization such as alcohols.

The term “pharmaceutical composition” as used herein refers to a mixture of a compound according to any of the embodiments described herein (such as compound (A) or a pharmaceutically acceptable salt thereof, compound (M) or a pharmaceutically acceptable salt thereof, or any combination of any of the foregoing) and one or more additional chemical components, such as, e.g., carriers, stabilizers, diluents, dispersing agents, suspending agents, thickening agents, and/or excipients.

The compounds (such as compound (A) or a pharmaceutically acceptable salt thereof, compound (M) or a pharmaceutically acceptable salt thereof) and pharmaceutical compositions according to any of the embodiments described herein can be administered by various routes of administration including, but not limited to, oral and parenteral administration.

The terms “effective amount” and “therapeutically effective amount,” as used herein, refer to a sufficient amount of an agent or a compound being administered which will relieve to some extent one or more of the symptoms of the disease or condition being treated. The result is reduction and/or alleviation of the signs, symptoms, or causes of a disease, or any other desired alteration of a biological system. For example, an “effective amount” for therapeutic use is the amount of a compound according to any embodiment described herein required to provide a clinically significant decrease in disease symptoms. In some embodiments, an appropriate “effective” amount in any individual case is determined using techniques such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “carrier,” as used herein, refers to relatively nontoxic chemical compounds or agents that facilitate the incorporation of a compound into cells or tissues.

The term “pharmaceutically acceptable carrier” or “pharmaceutically acceptable excipient” includes, but is not limited to, any and all solvents, dispersion media, coatings, antibacterial and antifungal agents, isotonic and absorption delaying agents, one or more suitable diluents, fillers, salts, disintegrants, binders, lubricants, glidants, wetting agents, controlled release matrices, colorants/flavoring, carriers, excipients, buffers, stabilizers, solubilizers, and combinations thereof. Except insofar as any conventional media or agent is incompatible with the active ingredient, its use in the therapeutic compositions of the invention is contemplated. Supplementary active ingredients can also be incorporated into the compositions.

As used herein, the terms “treatment,” “treating,” and “ameliorating” are used interchangeably. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “subject” or “patient” as used herein encompasses mammals and non-mammals. Examples of mammals include, but are not limited to, any member of the mammalian class, such as humans, non-human primates such as chimpanzees, and other apes and monkey species; farm animals such as cattle, horses, sheep, goats, and swine; domestic animals such as rabbits, dogs, and cats; and laboratory animals including rodents, such as rats, mice and guinea pigs. Examples of non-mammals include, but are not limited to, birds, fish and the like. In one embodiment of any of the methods, uses and compositions provided herein, the mammal is a human.

Suitable dual selective PI3K delta and gamma inhibitors include, but are not limited to, those having delta and gamma inhibition activity that is at least 50, 80, or 100 fold greater than for alpha and beta isoforms of PI3K. In one embodiment, the dual selective PI3K delta and gamma inhibitor is tenalisib or a pharmaceutically acceptable salt thereof. In another embodiment, the dual selective PI3K delta and gamma inhibitor is duvelisib or a pharmaceutically acceptable salt thereof.

SIK3 inhibitors are described in US 2021/0040486, which is hereby incorporated by reference. A preferred SIK3 inhibitor is a compound of formula (M) or a pharmaceutically acceptable salt thereof.

### Methods of Treatment and Uses

In any of the methods of treatment and uses described herein, one or more additional active agents can be administered with a compound of formula (A), or a pharmaceutically acceptable salt thereof. For example, the compound of formula (A), or a pharmaceutically acceptable salt thereof, can be administered in combination (administered together or sequentially) with known anti-cancer treatments such as, but not limited to, chemotherapy, radiation therapy, biological therapy, bone marrow transplantation, stem cell transplant or any other anticancer therapy or with one or more cytostatic, cytotoxic or anticancer agents or targeted therapy either alone or in combination, such as but not limited to, for example, DNA interactive agents, such as fludarabine, cisplatin, Chlorambucil, Bendamustine or doxorubicin; alkylating agents, such as, cyclophosphamide; topoisomerase II inhibitors, such as etoposide; topoisomerase I inhibitors such as CPT-11 or topotecan; tubulin interacting agents, such as paclitaxel, docetaxel, or the epothilones (for example ixabepilone), either naturally occurring or synthetic; hormonal agents, such as tamoxifen; aromatase inhibitors, such as anastrozole, letrozole, exemestane, and testolactone; selective estrogen receptor degraders or downregulators (SERD), such as Fulvestrant, Giredestrant, Amcenestrant, AZD9833, Rintodestrant, LSZ102, LY3484356, Elacestrant, ZN-c5, D-0502 and SHR9549; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors, Checkpoint kinase inhibitors, Salt induced kinase 3 (SIK3) inhibitors and monoclonal antibodies directed against growth factor receptors such as erbitux (EGF) and herceptin (Her2); CD20 monoclonal antibodies such as (HuMax; Intracel), AME-133v (LY2469298, Applied Molecular Evolution), and other anticancer agents such as Mundesine, Crizotinib, Romidepsin (Istodax), Belinostat, Pralatrexate (Folotyn), Gemcitabine, Alisertib (MLN8237), Dasatinib (Sprycel), E7777, Lenalidomide (Revlimid), Nelfinavir (Viracept), Panobinostat (LBH-589), Vorinostat (Zolinza), Everolimus (Afinitor), APO866, Carfilzomib (Kyprolis) and Mogamulizumab (KW-0761), or any combination of any of the foregoing.

In any of the methods of treatment and uses described herein, a compound of formula (A) or a pharmaceutically acceptable salt thereof can be administered with one or more additional active agents such as Mundesine, Crizotinib, Romidepsin (Istodax), Belinostat, Pralatrexate (Folotyn), Gemcitabine, Alisertib (MLN8237), Dasatinib (Sprycel), E7777, Lenalidomide (Revlimid), Nelfinavir (VIracept), Panobinostat (LBH-589), Vorinostat (Zolinza), Everolimus (Afinitor), APO866, Brentuximab Vedotin (Adcetris), Carfilzomib (Kyprolis), Mogamulizumab (KW-0761), or any combination of any of the foregoing.

In any of the methods of treatment and uses described herein, one or more additional active agents can be administered with a compound of formula (M) or a pharmaceutically acceptable salt thereof. For example, the compound of formula (M) or a pharmaceutically acceptable salt thereof, can be administered in combination (administered together or sequentially) with known anti-cancer treatments such as, but not limited to, chemotherapy, radiation therapy, biological therapy, bone marrow transplantation, stem cell transplant or any other anticancer therapy or with one or more cytostatic, cytotoxic or anticancer agents or targeted therapy either alone or in combination, such as but not limited to, for example, DNA interactive agents, such as fludarabine, cisplatin, Chlorambucil, Bendamustine or doxorubicin; Alkylating agents, such as, cyclophosphamide; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; aromatase inhibitors, such as anastrozole, letrozole, exemestane and testolactone; selective estrogen receptor degraders or downregulators (SERD), such as Fulvestrant, Giredestrant, Amcenestrant, AZD9833, Rintodestrant, LSZ102, LY3484356, Elacestrant, ZN-c5, D-0502 and SHR9549; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors, Checkpoint kinase inhibitors, Salt induced kinase 3 (SIK3) inhibitors and monoclonal antibodies directed against growth factor receptors such as erbitux (EGF) and herceptin (Her2); CD20 monoclonal antibodies such as (HuMax; Intracel),, AME-133v (LY2469298, Applied Molecular Evolution), and other anticancer agents such as Mundesine, Crizotinib, Romidepsin (Istodax), Belinostat, Pralatrexate (Folotyn), Gemcitabine, Alisertib (MLN8237), Dasatinib (Sprycel), E7777, Lenalidomide (Revlimid), Nelfinavir (VIracept), Panobinostat (LBH-589), Vorinostat (Zolinza), Everolimus (Afinitor), APO866, Carfilzomib (Kyprolis) and Mogamulizumab (KW-0761), or any combination of any of the foregoing.

In any of the methods of treatment and uses described herein, a compound of formula (M), or a pharmaceutically acceptable salt thereof, can be administered with one or more additional active agents such as Mundesine, Crizotinib, Romidepsin (Istodax), Belinostat, Pralatrexate (Folotyn), Gemcitabine, Alisertib (MLN8237), Dasatinib (Sprycel), E7777, Lenalidomide (Revlimid), Nelfinavir (VIracept), Panobinostat (LBH-589), Vorinostat (Zolinza), Everolimus (Afinitor), APO866, Brentuximab Vedotin (Adcetris), Carfilzomib (Kyprolis), Mogamulizumab (KW-0761), or any combination of any of the foregoing.

In any of the methods of treatment and uses described herein, a compound of formula (A) or a pharmaceutically acceptable salt thereof, and/or a compound of formula (M) or a pharmaceutically acceptable salt thereof, can be administered in combination with a radiation treatment.

In any of the methods of treatment and uses described herein, a compound of formula (A) or a pharmaceutically acceptable salt thereof, and/or a compound of formula (M) or a pharmaceutically acceptable salt thereof, can be administered in combination with surgery including either pre, post, or during period of surgery.

Any of these treatments can be administered simultaneously, separately, sequentially and/or spaced in time.

### Pharmaceutical Compositions

The present invention provides a pharmaceutical composition comprising a Compound of formula (A) or a pharmaceutically acceptable salt thereof. The pharmaceutical composition may include one or more additional active ingredients as described herein. The pharmaceutical composition may be administered for any of the disorders described herein.

In one embodiment, the pharmaceutical composition comprises a therapeutically effective amount of a compound of formula (A) or a pharmaceutically acceptable salt thereof, as the active ingredient. Where desired, the pharmaceutical compositions contain a compound of formula (A) or a pharmaceutically acceptable salt thereof, as the active ingredient and one or more pharmaceutically acceptable excipients, carriers, such as inert solid diluents and fillers, diluents, including sterile aqueous solution and various organic solvents, permeation enhancers, solubilizers and adjuvants, or any combination of any of the foregoing.

The present invention also provides a pharmaceutical composition comprising a compound of formula (M) or a pharmaceutically acceptable salt thereof. The pharmaceutical composition may include one or more additional active ingredients as described herein. The pharmaceutical composition may be administered for any of the disorders described herein.

In another embodiment, the pharmaceutical composition comprises a therapeutically effective amount of compound of formula (M) or a pharmaceutically acceptable salt thereof, as the active ingredient. Where desired, the pharmaceutical compositions contain a Compound of formula (M), or a pharmaceutically acceptable salt thereof, as the active ingredient and one or more pharmaceutically acceptable excipients, carriers, such as inert solid diluents and fillers, diluents, including sterile aqueous solution and various organic solvents, permeation enhancers, solubilizers and adjuvants, or any combination of any of the foregoing.

The compounds and pharmaceutical compositions described herein can be administered alone or in combination with one or more other agents, which are also typically administered in the form of a pharmaceutical composition. Where desired, the compounds and compositions described herein and other agent(s) may be mixed into a preparation or both components may be formulated into separate preparations to use them in combination separately or at the same time.

Methods and uses described herein include administration of an inhibitor according to any embodiment described herein by itself, or in combination as described herein, and in each case optionally including one or more suitable diluents, fillers, salts, disintegrants, binders, lubricants, glidants, wetting agents, controlled release matrices, colorants/flavoring, carriers, excipients, buffers, stabilizers, solubilizers, and combinations thereof. Preparations of various pharmaceutical compositions are known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2003; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999), all of which are incorporated by reference herein in their entirety.

The compounds and compositions according to any of the embodiments described herein can be administered by any route that enables delivery of the compounds to the site of action, such as oral routes, intraduodenal routes, parenteral injection (including intravenous, intraarterial, subcutaneous, intramuscular, intravascular, intraperitoneal or infusion), topical administration (e.g. transdermal application), rectal administration, via local delivery by catheter or stent or through inhalation. The compounds and compositions according to any of the embodiments described herein can also be administered intraadiposally or intrathecally.

The compositions according to any of the embodiments described herein can be administered in solid, liquid or in dried powder, such as lyophilized form. The pharmaceutical compositions can be packaged in forms convenient for delivery, including, for example, solid dosage forms such as capsules, sachets, cachets, gelatins, papers, tablets, capsules, suppositories, pellets, pills, troches, and lozenges. The type of packaging will generally depend on the desired route of administration. Implantable sustained release formulations are also contemplated, as are transdermal formulations.

The following general methodology described herein provides the manner and process of using the compound of the present invention and are illustrative rather than limiting. Further modification of provided methodology and additionally new methods may also be devised in order to achieve and serve the purpose of the invention. Accordingly, it should be understood that there may be other embodiments which fall within the spirit and scope of the invention as defined by the specification hereto.

### EXAMPLES

### Example 1

### Composition of Compound of Formula (A)

Tenalisib (Compound of Formula (A)) is available as tablets of 200 and 400 mg strength. An exemplary composition comprising tenalisib is shown in Table 1.

TABLE 1
Exemplary Composition of Tenalisib

| S. No. | Ingredient | % w/w |
|---|---|---|
| 1 | Compound of Formula (A) (Tenalisib) | 50.00 |
| 2 | Microcrystalline cellulose (Avicel PH102) | 40.25 |
| 3 | Hydroxypropyl cellulose (Klucel LF) | 2.00 |
| 4 | Purified Water | — |
| 5 | Croscarmellose Sodium (AC-DI-SOL) | 6.00 |
| 6 | Talc | 1.00 |
| 7 | Colloidal silicon dioxide (Aerosil-200) | 0.25 |
| 8 | Magnesium stearate | 0.50 |
| 9 | Opadry 03F520226 Yellow | 3.0 |

TABLE 2
Exemplary Composition of Tenalisib
for 200 mg and 400 mg strengths

| Strength | | | 200 mg | 400 mg |
|---|---|---|---|---|
| S. No. | Ingredient | Specs. | mg/tab | |
| I | Intragranular Part | | | |
| a | Dry Mix | | | |
| 1 | Compound of Formula (A) | IH | 200.00 | 400.00 |
| 2 | Microcrystalline Cellulose (Avicel | NF/ | 161.00 | 322.00 |
| | PH 102) | Ph. Eur. | | |
| b | Binder Solution | | | |
| 4 | Hydroxypropyl Cellulose (Klucel | NF/ | 8.00 | 16.00 |
| | LF) | Ph. Eur. | | |
| 5 | Purified Water | USP | q.s. | q.s. |
| II | Extra-granular Part | | | |
| c | Blending/Mixing | | | |
| 6 | Croscarmellose Sodium (Ac-Di- | NF/ | 24.00 | 48.00 |
| | Sol) | Ph. Eur. | | |
| 7 | Talc (Purified) | USP/ | 1.00 | 2.00 |
| | | Ph. Eur. | | |
| 8 | Colloidal Silicon Dioxide (Aerosil | NF/ | 4.00 | 8.00 |
| | 200) | Ph. Eur. | | |
| d | Lubrication | | | |
| 9 | Magnesium Stearate | NF/ | 2.00 | 4.00 |
| | | Ph. Eur. | | |
| | Total weight of Lubricated Blend | | 400.00 | 800.00 |
| III | Coating | | | |
| 10 | Opadry Yellow 03F520226 | IH | 12.00 | 24.00 |
| 11 | Purified Water | IH | q.s. | q.s. |
| | Total weight of coated tablets | | 412.00 | 824.00 |

### Manufacturing Process:

1. Dispense raw material quantities as outlined in the above table.
2. Co-sift API (Compound of formula (A)) with microcrystalline cellulose (Avicel PH 102) through #40 mesh sieve.
3. Dissolve the weighed quantity of hydroxypropyl cellulose (Klucel LF) in purified water under stirring in a stainless-steel container to obtain a clear solution.
4. Granulate the blend of step 2 with the binder solution of step 3 in a rapid mixer granulator (RMG).
5. Dry the wet mass of step 4 in a rapid dryer at an inlet temperature of 65° C.
6. Pass the dried granules of step 5 through #24 mesh sieve manually and mill oversized granules through multi-mill equipped with a 1.0 mm screen with medium speed and knives forward direction. Sift the milled granules again through 24 #Mesh.
7. Sift croscarmellose sodium (Ac-di-sol SD 711), colloidal silicon dioxide (Aerosil 200) and talc (purified) through #40 mesh sieve and mix with the granules of step 6 in a blender for 10 mins at 12 RPM.
8. Sift magnesium stearate through #60 mesh and mix with the blend of step 7 in a blender for 6 mins at 12 RPM.
9. Compress the lubricated blend with a suitable punch.
10. Disperse Opadry Yellow 03F520226 in purified water with stirring in a stainless-steel container.
11. Coat the compressed tablets with the Opadry dispersion of step 9 until 3% weight gain is achieved in a pan coater.

### Example 2

### Pharmacokinetics

Pharmacokinetic studies following single-dose and repeat dose administration were performed in mice, rats, dogs, humans and monkeys. Tenalisib plasma concentrations were determined by a validated LC-MS/MS method. The pharmacokinetic profile of tenalisib was evaluated following both oral and intravenous administration. Absorption of tenalisib across the tested species was found to be rapid with moderate clearance and an oral bioavailability of >72.4% in rats, 21.6% in mice and >100.0% in dogs. Tenalisib was found to have high protein binding (>96%) in animals as well as in human plasma. In an in vitro study using liver microsomes, the metabolic stability followed a rank order of rat>human>dog>monkey>mouse.

### Distribution

### Protein Binding

In vitro protein binding studies were performed using rat, dog, monkey and human plasma. Binding of tenalisib to plasma proteins was >96% across all species.

### Plasma Stability

In vitro plasma stability studies were performed using rat, dog, monkey and human plasma. The concentration of tenalisib was measured by LC-MS/MS. Tenalisib was found to be stable across all species in plasma.

### Tissue Distribution

Tissue distribution upon single oral administration (10 mg/kg bodyweight) of tenalisib was studied in male wistar rats. The rank order of Cmax and AUC in Wistar rats was liver>kidney>heart>plasma>thymus>spleen>serum>lung>blood>brain.

### Metabolism

In in vitro studies using liver microsomes, the metabolic stability of tenalisib followed the rank order of rat>human>dog>monkey>mouse (Table 3). Tenalisib (Compound A) and Compound M demonstrated >50% metabolic stability in rat, dog, and human and <30% in mouse and monkey liver microsomes.

TABLE 3
Summary of Metabolic Stability Across Species

% Metabolic Stability

| S. No. | Species | Compound A | Compound M |
|---|---|---|---|
| 1 | Mouse | 26.0 | 9.1 |
| 2 | Rat | 79.3 | 58.0 |
| 3 | Dog | 52.2 | 67.6 |
| 4 | Monkey | 29.6 | 1.2 |
| 5 | Human | 73.9 | 58.5 |

### Example 3

### PI3K δ/γ and SIK3 Inhibition Activity

The compound of formula (A) is a dual PI3K δ/γ inhibitor and a selective SIK3 inhibitor with nano-molar potency against both PI3K δ/γ isoforms in enzyme, cell, and blood-based assays while retaining several fold selectivity over PI3K α and B. The compound of formula (M) inhibited SIK3 with an IC₅₀ of 237 nM. A summary of in vitro enzyme and cell-based assays is presented in Table 4.

TABLE 4
Summary of In Vitro Enzyme and Cell Based Assays

| Study | Procedure | Results | Conclusion |
|---|---|---|---|
| PI3K isoform | HTRF based assay in the | IC50 of PI3Kδ = 24.5 nM | Highly |
| enzyme assay | presence of 100 μM ATP | IC50 of PI3Kγ = 33.2 nM | selective |
| | | IC50 of PI3Kβ = 4023 nM | over the α and β |
| | | IC50 of PI3Kα = >1 0000 nM | isoforms |
| PI3K isoform | PI3K α or β-mediated | EC50 of PI3Kδ = 38.1 nM | Highly |
| Cell-based | phosphorylation of AKT by | EC50 of PI3Kγ = 22.3 nM | selective |
| assay | platelet-derived growth factor | EC50 of PI3Kβ = 2067 nM | over the α and β |
| | (PDGF) or lysophosphatidic | EC50 of PI3Kα = >10000 nM | isoforms |
| | acid in 3T3 fibroblasts; c5a | | |
| | induced pAKT in RAW | | |
| | macrophages for PI3Kγ; anti- | | |
| | IgM induced human B-cell | | |
| | proliferation for PI3Kδ | | |
| SIK3 | Enzymatic radiometric assay | IC50 = 237 nM | Selective over |
| inhibition | in presence of 10 μM ATP | | the SIK1 and |
| assay | | | SIK2 isoforms |
| fMLP or anti | Whole blood samples | EC50 of anti-FcεR1 (PI3Kδ) | Potent activity |
| FcεR1 induced | collected from healthy human | mediated CD63 expression = | against both |
| CD63 | subjects were treated with | 39.0 nM | PI3Kδ and γ- |
| expression | DMSO or tenalisib prior to | EC50 of fMLP (PI3Kγ) | mediated |
| | activation with anti FcεR1 | mediated CD63 expression = | signaling |
| | mAb or fMLP. Cells were | 37.8 nM | |
| | stained with a combination | | |
| | of anti-human CD63-FITC | | |
| | and anti-human CCR3-PE | | |
| | mAbs and analyzed by flow | | |
| | cytometry. | | |

### Example 4

Assay 1: Combination of Compound A and Compound M with Fulvestrant in Zr.75.1 Cell Lines (without B-Estradiol)

### Procedure

### Cell Proliferation:

Zr.75.1 cells were plated in their Roswell Park Memorial Institute (RPMI-1640)+10% FBS (without B-estradiol) media at pre-determined density in a 96-well plate.
Cells were exposed to either DMSO (control) alone, Compound A+Compound M, fulvestrant alone, or a combination of Compound A+Compound M and fulvestrant.
Cells were incubated for 72 hours in presence of the inhibitors.
MTT reaction was performed to determine the cell viability at the end of the treatment period and absorbance was measured at wavelength 560 and 640 nM.
Cell growth inhibitions were calculated, and Tukey's analysis was performed using GraphPad Prism to compare the treated groups.

### Cell Cycle Analysis:

Zr.75.1 cells were plated in their RPMI 1640+10% FBS (without β-estradiol) media at pre-determined density in a 6-well plate.
Cells were exposed to either DMSO (control) alone, or Compound A+Compound M, fulvestrant alone, or a combination of Compound A+Compound M and fulvestrant.
Cells were incubated for 48 hours in presence of the inhibitors.
At the end of the treatment period cells were pelleted, and the supernatant was discarded.
The cell pellet was washed with PBS at 250× g for 5 minutes.
The washed pellet was resuspended in 1×PBS.
The suspension was added to 1 mL of ice cold 70% Ethanol in a drop wise manner and stored at −20° C. for 3 h up to a week.
On the day of cell cycle analysis, cells were centrifuged at 1200 rpm for 5 min and supernatant discarded.
Cells were washed with of PBS and centrifuged at 1200 rpm for 5 min and supernatant discarded.
The pellet was re-suspended in PBS and cell cycle reagent was added and mixed well. Samples were incubated at room temperature for 30 minutes in the dark.
Samples were acquired and analysed on a MUSE cell analyzer.
positive populations were plotted using GraphPad Prism.

### Results

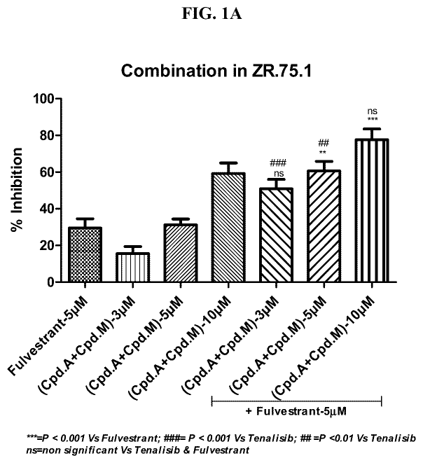

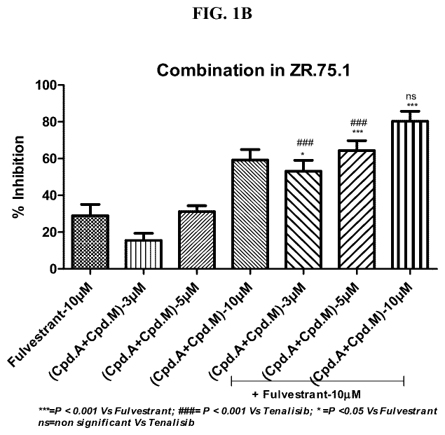

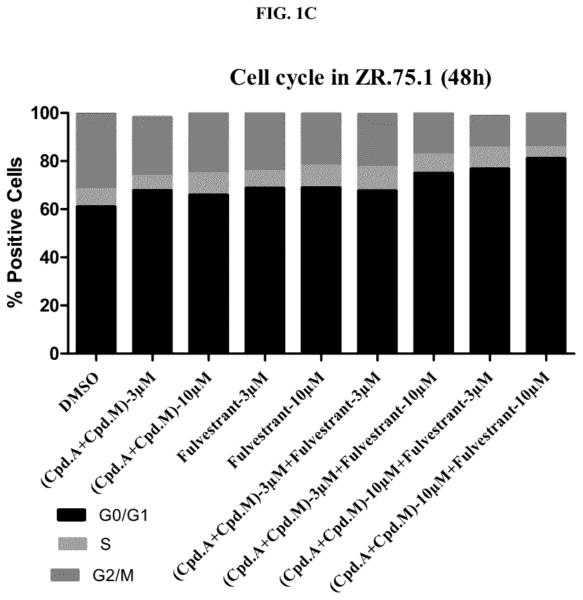

Compound A+Compound M significantly potentiated the activity of fulvestrant (5 M and 10 μM) in inhibiting the cell growth in ZR.75.1 breast cancer cell line in absence of estrogen.
Combination treatment with Fulvestrant and Compound A+Compound M induced >14% (2-fold increase) G0/G1 cell cycle arrest in ZR.75.1 breast cancer cell line in the absence of estrogen. The results are shown in FIGS. 1A, 1B and 1C.

### Example 5

Assay 2: Combination of Compound A+Compound M with Fulvestrant in Zr.75.1 Cell Lines (with β-Estradiol)

### Procedure:

### Cell Proliferation:

Zr.75.1 cells were plated in their RPMI+10% FBS (without β-estradiol) media at pre-determined density in a 96-well plate.
Cells were exposed to either DMSO (control) alone, Compound A+Compound M, Fulvestrant alone, or a combination of Compound A+Compound M and Fulvestrant.
Cells were incubated for 96 h in presence of the inhibitors.
MTT reaction was performed to determine the cell viability at the end of the treatment period and absorbance was measured at wavelength 560 and 640 nM.
% cell growth inhibitions were calculated, and Tukey's analysis was performed using GraphPad Prism to compare the treated groups.

### Cell Cycle Analysis:

Zr.75.1 cells were plated in their RPMI+10% FBS (without B-estradiol) media at pre-determined density in a 6-well plate.
Cells were exposed to either DMSO (control) alone, Compound A+Compound M, fulvestrant alone, or a combination of Compound A+Compound M and fulvestrant.
Cells were incubated for 48 h in presence of the inhibitors.
At the end of the treatment period cells were pelleted, and supernatant was discarded.
Cell pellet was washed with PBS at 250× g for 5 minutes.
The washed pellet was resuspended in 1×PBS.
The suspension was added to 1 mL of ice cold 70% Ethanol in a drop wise manner and stored at −20° C. for 3 hours up to a week.
On the day of cell cycle analysis, cells were centrifuged at 1200 rpm for 5 minutes and supernatant discarded.
Cells were washed with PBS and centrifuged at 1200 rpm for 5 minutes and the supernatant discarded.
Pellet was re-suspended in PBS and cell cycle reagent was added and mixed well. Samples were incubated at room temperature for 30 minutes in the dark.
Samples were acquired and analysed on a MUSE cell analyzer.
% positive populations were plotted using GraphPad Prism.

### Results

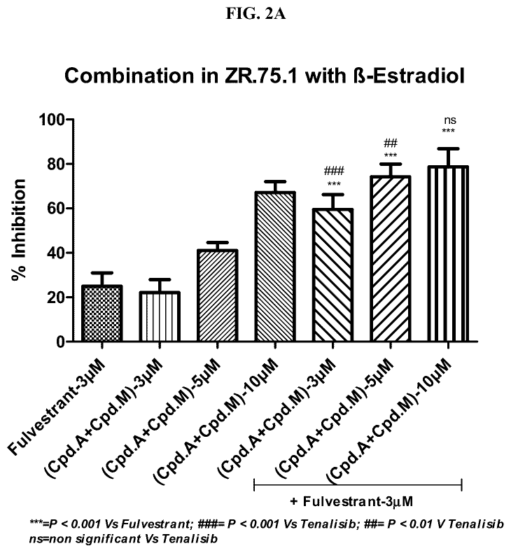

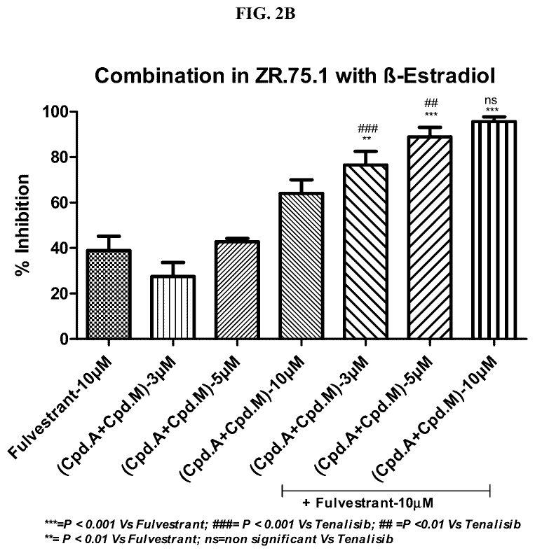

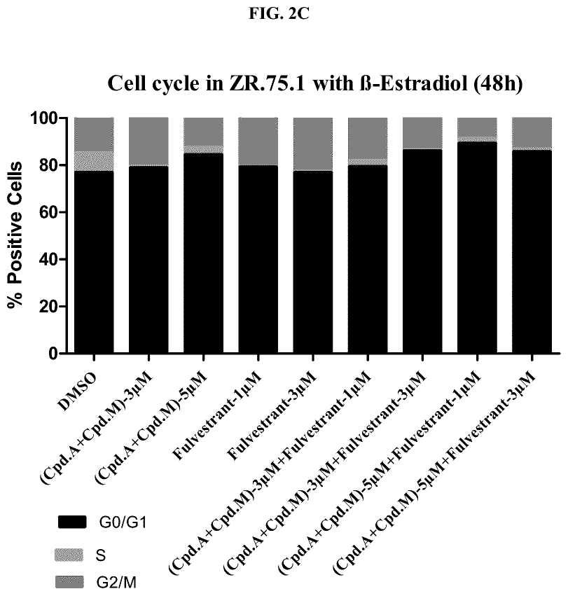

The combination of Compound A+Compound M significantly potentiated the activity of fulvestrant (3 μM and 10 μM) in inhibiting the cell growth in ZR.75.1 breast cancer cell lines in presence of estrogen.
Combination treatment with fulvestrant (3 μM) with Compound A+Compound M (3 μM) induced ˜10% (3-fold increase) G0/G1 cell cycle arrest in ZR.75.1 breast cancer cell lines in presence of estrogen. The results are shown in FIGS. 2A, 2B and 2C.

### Example 6

Assay 3: Combination of Compound A+Compound M with Fulvestrant in MCF-7 Cell Lines (with β-Estradiol)

### Procedure

### Cell Proliferation:

MCF-7 cells were plated in their minimum essential medium (MEM)+10% FBS (with β-estradiol) media at pre-determined density in a 96-well plate.
Cells were exposed to either DMSO (control) alone, Compound A+Compound M, fulvestrant alone, or a combination of Compound A+Compound M and fulvestrant.
Cells were incubated for 96 hours in presence of the inhibitors.
MTT reaction was performed to determine the cell viability at the end of the treatment period and absorbance was measured at wavelength 560 and 640 nM.
% cell growth inhibitions were calculated, and Tukey's analysis was performed using GraphPad Prism to compare the treated groups.

### Cell Cycle Analysis:

MCF-7 cells were plated in their MEM+10% FBS (with β-estradiol) media at pre-determined density in a 6-well plate.
Cells were exposed to either DMSO (control) alone, Compound A+Compound M, fulvestrant alone, or a combination of Compound A+Compound M and fulvestrant.
Cells were incubated for 48 h in presence of the inhibitors.
At the end of the treatment period cells were pelleted, and supernatant was discarded.
Cell pellet was washed with PBS at 250× g for 5 minutes.
The washed pellet was resuspended in 1×PBS.
The suspension was added to 1 mL of ice cold 70% ethanol in a drop wise manner and stored at −20° C. for 3 hours up to a week.
On the day of cell cycle analysis, cells were centrifuged at 1200 rpm for 5 minutes and the supernatant discarded.
Cells were washed with PBS 250× g?? and centrifuged at 1200 rpm for 5 minutes and the supernatant discarded.
Pellet was re-suspended in PBS and cell cycle reagent was added and mixed well.
Samples were incubated at room temperature for 30 minutes in the dark.
Samples were acquired and analysed on a MUSE cell analyzer.
% positive populations were plotted using GraphPad Prism.

### Results:

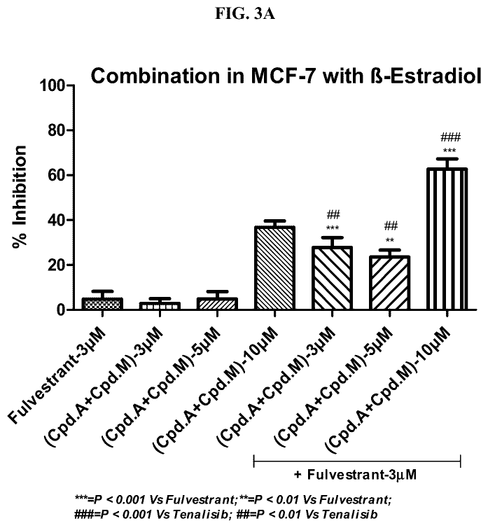

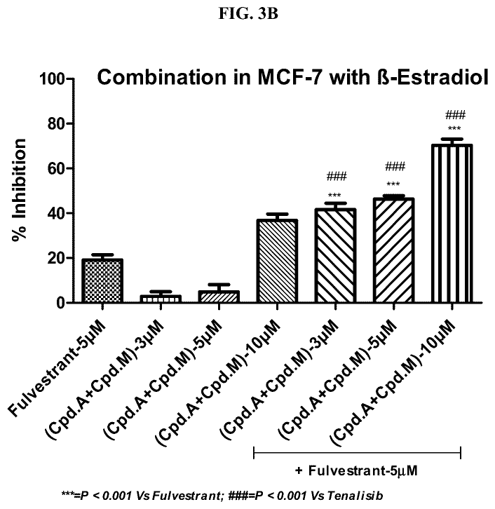

The combination of Compound A+Compound M significantly potentiated the activity of fulvestrant (3 μM,5 μM) in inhibiting the cell growth in MCF-7 breast cancer cell lines in presence of estrogen. The results are shown in FIGS. 3A and 3B.

### Example 7

Assay 4: Combination of Compound A+Compound M with/without Standard of Care (SOC) in Breast Cancer Cell Lines (MDA-MB-231 and MCF-7)

### Procedure

### Cell Proliferation:

Breast Cancer cells were plated in their respective complete media at pre-determined density in a 96-well plate.
Cells were exposed to either DMSO (control) alone, Compound A alone, Compound M alone, taxol/doxorubicin alone, or a combination of Compound A+Compound M and taxol/doxorubicin.
Cells were incubated for 72 h in presence of the inhibitors.
MTT reaction was performed to determine the cell viability at the end of the treatment period and absorbance was measured at wavelength 560 and 640 nM.
% cell growth inhibitions were calculated, and Tukey's analysis was performed using GraphPad Prism to compare the treated groups.

### Results:

Taxol alone showed an anti-proliferative effect by inhibiting the growth by 34.5% and 28.8% in MDA-MB-231 and MCF-7 breast cancer cell lines, respectively. Addition of Compound A and Compound M (at 5 μM each) in combination potentiated the activity of taxol in breast cancer cell lines with a percent growth inhibition of 49.3% and 49.2% in MDA-MB-231 and MCF-7 cell lines, respectively. The combination of Compound A and Compound M potentiated the activity of taxol in attenuating the growth of breast cancer cell lines.

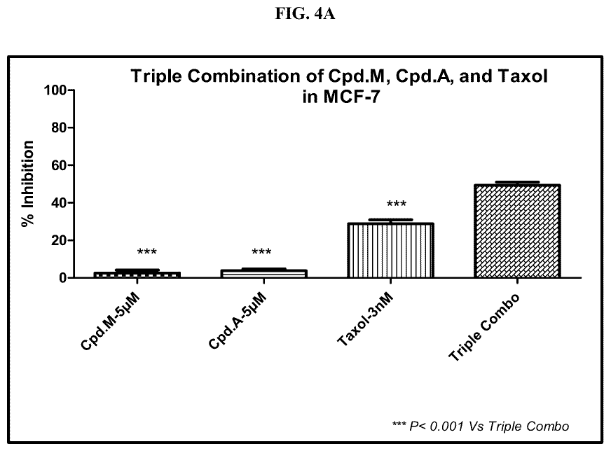

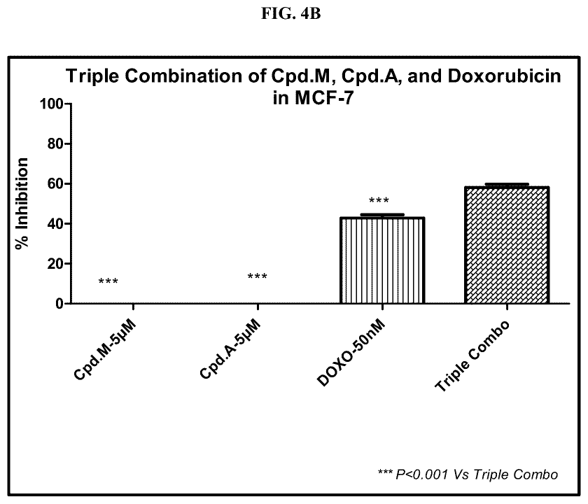

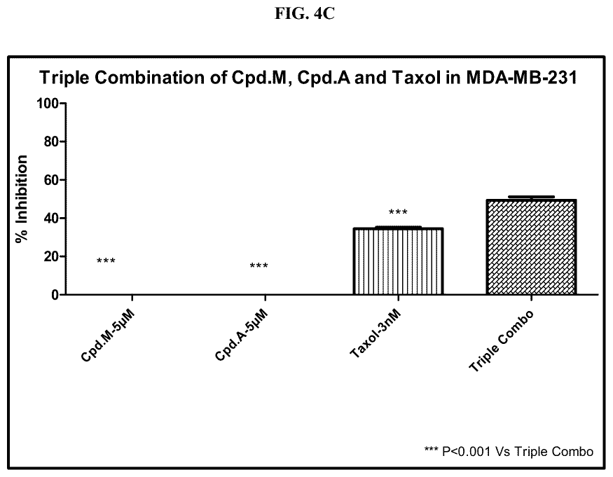

Doxorubicin alone or in combination with Compound A and Compound M (at 5 μM each) showed 42.8% and 58.1% growth inhibition in MCF-7 breast cancer cell lines, respectively, indicating that the combination of Compound A and Compound M potentiated the activity of doxorubicin in this cell line. The results are shown in FIGS. 4A, 4B and 4C.

### Example 8

### Assay 5: Combination of Compound M and Olaparib in Breast Cancer Cell Lines (MCF-7 Cell Lines)

### Procedure

### Cell Proliferation:

MCF-7 cells were plated in MEM+10% FBS complete media at pre-determined density in a 96-well plate.
Cells were exposed to either DMSO (control) alone, Compound M alone, olaparib alone, or a combination of Compound M and olaparib.
Cells were incubated for 72 h in presence of the inhibitors.
MTT reaction was performed to determine the cell viability at the end of the treatment period and absorbance was measured at wavelength 560 and 640 nM.
% cell growth inhibitions were calculated, and Tukey's analysis was performed using GraphPad Prism to compare the treated groups.

### Results:

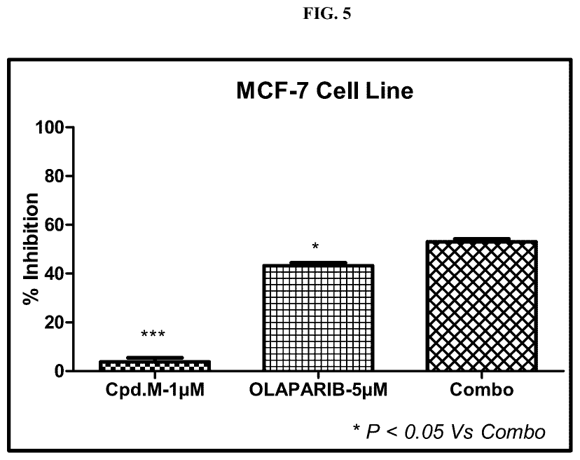

In breast cancer MCF-7 cell lines, Olaparib alone or in combination with Compound M (1 μM) showed 43.2% and 53.0% growth inhibition, respectively. The results are shown in FIG. 5.

### Example 9

Anti-Proliferative Activity of Compound M in Combination with PARP Inhibitors in Ovarian Cancer Cell Lines

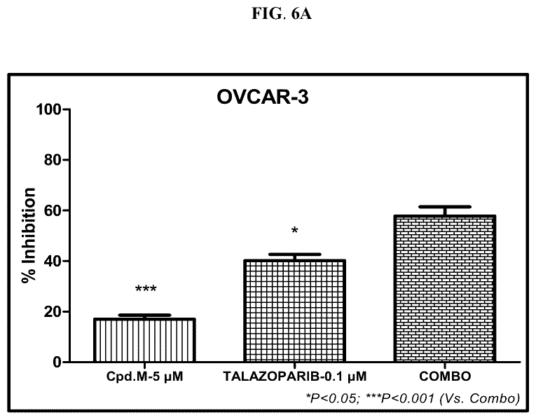

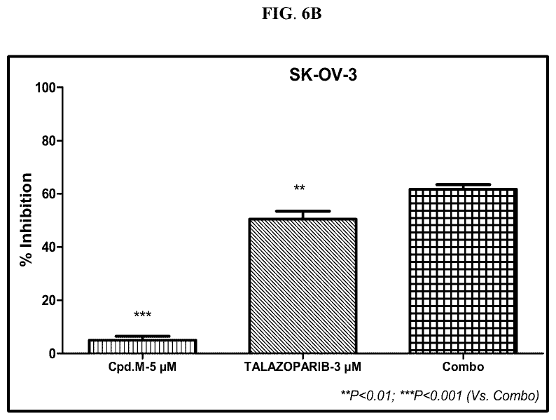

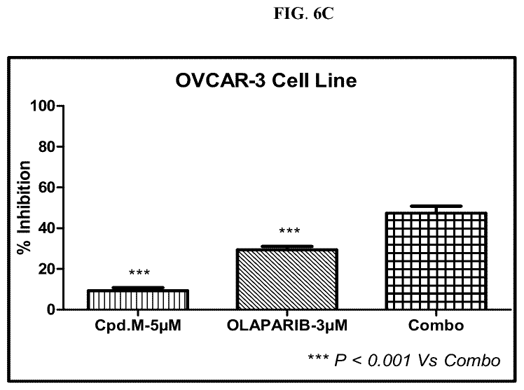

Single agent talazoparib demonstrated an anti-proliferative effect (inhibits the growth by 40.23% and 50.0% in OVCAR-3 and SK-OV-3 ovarian cancer cell lines, respectively). Addition of Compound (M) (5 μM) potentiated the activity of talazoparib in ovarian cancer cell lines and the percent growth inhibition was 57.8% and 61.6% in OVCAR-3 and SK-OV-3 cell lines, respectively. This is shown in FIGS. 6A, 6B and 6C.

Olaparib demonstrated anti-proliferative effect by inhibiting the growth by 29.3% in OVCAR-3 cell line.

### Example 10

Activity of Compound of Formula (M) Alone and in Combination with Taxol in Ovarian Cancer Cell Lines

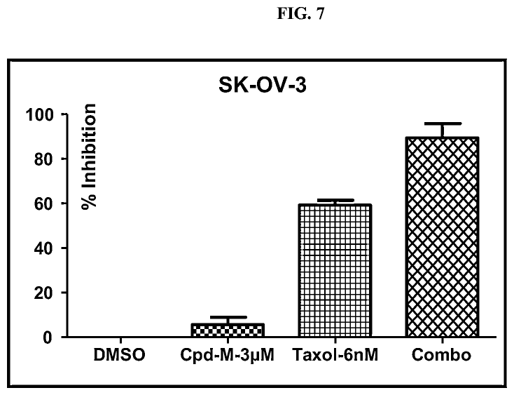

Taxol demonstrated an anti-proliferative effect by inhibiting the growth by 44.4%, 25.8%, 17.3% and 59.2% in breast (MDA-MB-231, ZR.75.1, and MCF-7) and ovarian (SK-OV-3) cancer cell lines respectively. Addition of Compound (M) (3 μM) increased taxol induced growth in ovarian (SK-OV-3) cancer cell line. This indicates that Compound (M) potentiated the activity of taxol. This is shown in FIG. 7.

### Example 11

Combination of Compound of Formula (M) with Compound of Formula (A) in Pancreatic Cancer Cell Lines

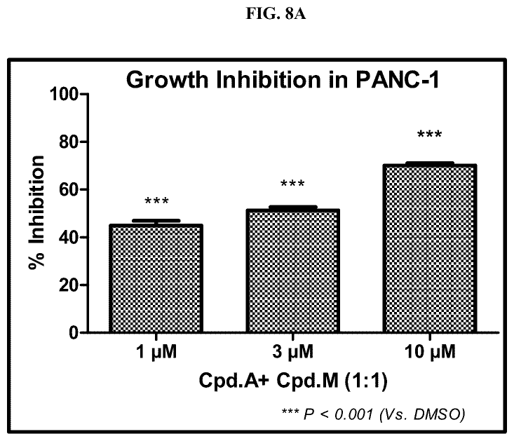

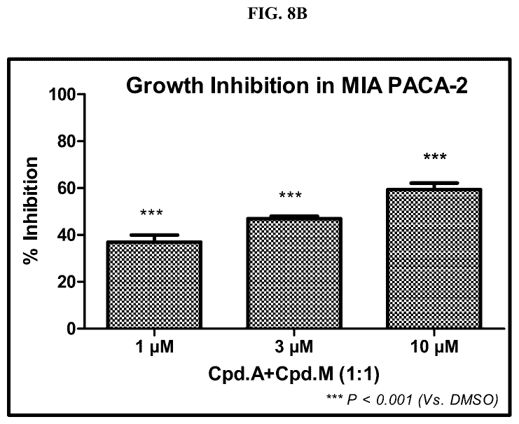

A combination of Compound of formula (M) with Compound of formula (A) at a 1:1 mole ratio demonstrated anti-proliferative effect by inhibiting the growth by 44.9%, 51.2%, and 70.05% in pancreatic (PANC-1) cancer cell line and by 36.9%, 46.9%, and 59.4% in pancreatic (MIA paca-2) cancer cell lines at 1, 3 & 10 μM, respectively. This indicates that the combination of Compound of formula (A) and Compound of formula (M) is effective in pancreatic cancer. This is shown in FIGS. 8A and 8B.

### Example 12

Efficacy Evaluation of Tenalisib Alone and in Combination with Anti-PD1 in the MC38 Syngeneic Murine Colon Carcinoma C57BL/6 Model

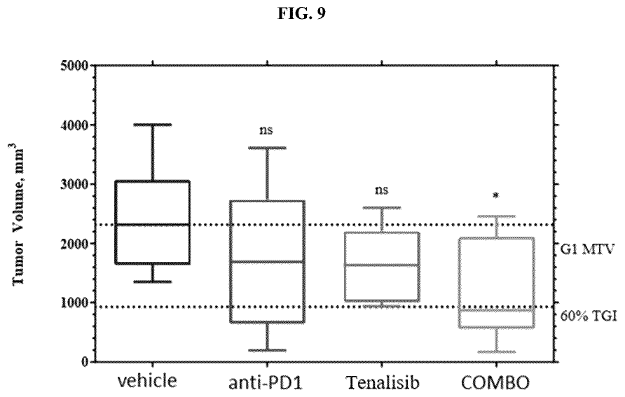

Daily tenalisib alone and in combination with anti-PD-1 (clone: RMP1-14) was evaluated for tumor growth inhibition (TGI) in the MC38 syngeneic murine colon carcinoma model in immunocompetent C57BL/6 female mice. Treatment with tenalisib resulted in a 29% TGI (Tumour Growth Inhibition) (at day 22) compared to control. Treatment with the combination (tenalisib+anti-PD-1) resulted in 63% TGI (P<0.05) when compared to control. This is shown in FIG. 9.

### Example 13

### Effect of Compound A on Breast Cancer Patients

### Trial Design

This is a Phase II, randomized, open label study, designed to evaluate the preliminary efficacy and safety of tenalisib (Compound A) at two dose levels (800 mg BID and 1200 mg BID) in 40 patients with locally advanced or metastatic breast cancer. 20 patients will be enrolled in each of Group 1 (tenalisib 800 mg BID) and Group 2 (tenalisib 1200 mg BID). Both groups will be run in parallel.

### Primary Objective

To assess the anti-tumour activity of tenalisib in patients with locally advanced or metastatic breast cancer

### Secondary Objective

To evaluate the safety and tolerability of tenalisib.

### Exploratory Objectives

Changes in serum cytokines/chemokines and tumour tissue gene expression post treatment with tenalisib.

### Key Eligibility Criteria

1. Provision of full informed consent prior to any study-specific procedures.
2. Patients must be ≥18 years of age, at the time of signing informed consent.
3. Female patients who have histologically and/or cytologically confirmed locally advanced or metastatic breast cancer that has progressed following at least one line of therapy.
4. Patients with at least one measurable lesion per RECIST version 1.1 at baseline that can be accurately assessed by CT scan or MRI and is suitable for repeated assessment at follow up-visits.
5. ECOG performance status 0 to 2.
6. Life expectancy of at least 3 months.
7. Adequate bone marrow, liver and renal functions as assessed within 7 (+2) days before the first dose of study drug with the following laboratory requirements:
   Haemoglobin ≥8.0 g/dL (should not be transfused or treated with erythropoietin to maintain or exceed this level).
   Absolute neutrophil count (ANC) ≥1.0×109/L without growth factor support
   Platelet count ≥75×109/L.
   Total bilirubin≤1.5 times the ULN (or ≤3× ULN, if the patient has Gilbert syndrome).
   ALT and AST≤3× ULN (≤5× ULN for patients with liver metastasis or liver involvement).
   Creatinine clearance ≥50 mL/min (calculated using the Cockcroft-Gault formula) for patients with creatinine levels above ULN.
8. Female patient of childbearing potential should be willing to use a medically acceptable method of contraception as defined in Appendix B while participating in the study and for 30 days after the last dose of study drug AND must have a negative serum pregnancy test within 3 days prior to Cycle 1 Day 1 (C1D1).
9. Ability to swallow and retain oral medication.
10. Willingness and capability to comply with the study requirements.

The patient demographics are provided below.

### Patient Demographics

| | 800 mg | | 1200 mg BID | | All | |
|---|---|---|---|---|---|---|
| Parameters | BID(n = 20) | | (n = 20) | | (n = 40) | |
| Age, years, Median (range) | 61 | (31-71) | 64 | (51-71) | 63 | (31-71) |
| Median time from initial | 47 | (7-179) | 59 | (6-195) | 49 | (6-195) |
| diagnosis to first dose of | | | | | | |
| tenalisib, months (range) | | | | | | |
| de novo metastasis n (%) | 12 | (60) | 8 | (40) | 20 | (50) |

| | | | |
|---|---|---|---|
| Stage IVA/IVB, n | 7/13 | 7/13 | 14/26 |

Site of disease/metastasis, n

| | | | | | | |
|---|---|---|---|---|---|---|
| Visceral Disease | 13 | (65) | 18 | (90) | 31 | (77.5) |
| Liver | 8 | (40) | 9 | (45) | 17 | (42.5) |
| Lung | 8 | (40) | 12 | (60) | 20 | (50) |
| Breast | 14 | (70) | 6 | (30) | 20 | (50) |
| Lymph node | 14 | (70) | 14 | (70) | 28 | (70) |
| Bone | 12 | (60) | 13 | (65) | 25 | (62.5) |

No. of metastatic sites, n

| | | | | | | |
|---|---|---|---|---|---|---|
| ≥3 | 16 | (80) | 14 | (70) | 30 | (75) |

| | | | |
|---|---|---|---|
| ECOG status, 0/1/2, n | 10/10/0 | 8/12/0 | 18/22/0 |

Subtypes, n

| | | | |
|---|---|---|---|
| ER+, PR+/ER+, PR−/ | 15/5/0 | 16/3/1 | 31/8/1 |
| TNBC | | | |

Prior systemic therapies given in any setting

| | | | | | | |
|---|---|---|---|---|---|---|
| Median no. of prior | 3 | (1-6) | 3 | (1-7) | 3 | (1-7) |
| therapies (range) | | | | | | |

Prior therapies given in advanced/metastatic setting

| | | | | | | |
|---|---|---|---|---|---|---|
| Median no. of prior | 2 | (0-5) | 1 | (1-5) | 2 | (0-5) |
| therapies (range) | | | | | | |
| Median no. of prior | 2 | (0-3) | 1 | (0-2) | 1 | (0-3) |
| endocrine therapies, (range) | | | | | | |
| Patients who received | 9 | (45) | 10 | (50) | 19 | (47.5) |
| chemotherapy, n (%) | | | | | | |

Last Prior Therapy

| Aromatase inhibitors | 9 | (45) | 8 | (40) | 17 | (42.5) |
|---|---|---|---|---|---|---|
| Tamoxifen | — | | 1 | (5) | 1 | (2.5) |
| Fulvestrant | 7 | (35) | 5 | (25) | 12 | (30) |
| Chemotherapy | 5 | (25) | 6 | (30) | 11 | (27.5) |

Endocrine resistance status^{#}

| | | | | | | |
|---|---|---|---|---|---|---|
| Primary Resistance, n (%) | 7 | (35) | 5 | (28) | 12 | (30) |
| Secondary Resistance, n | 13 | (65) | 13 | (72) | 26 | (65) |
| (%) | | | | | | |

### Efficacy Data:

| | 800 mg BID | | 1200 mg BID | | All | |
|---|---|---|---|---|---|---|
| Parameters | (N = 20) | | (N = 20) | | (N = 40) | |
| Objective Response rate (ORR), | 2 | (10) | 3 | (15) | 5 | (12.5) |
| n(%) | | | | | | |
| Complete Response, n(%) | — | | — | | — | |
| Partial Response, n(%) | 2 | (10) | 3 | (15) | 5 | (12.5) |
| Stable disease, n(%) | 11 | (55) | 11 | (55) | 22 | (55) |
| Progressive disease, n(%) | 5 | (25) | 4 | (20) | 9 | (22.5) |
| Not Assessed, n (%) | 2 | (10) | 2 | (10) | 4 | (10) |
| SD ≥24 weeks, n (%) | 9 | (45) | 9 | (45) | 18 | (45) |
| Clinical Benefit Rate, n (%) | 11 | (55) | 12 | (60) | 23 | (57.5) |
| (CBR = PR + CR + SD ≥24 weeks) | | | | | | |
| Disease Control rate (DCR), n (%) | 13 | (65) | 14 | (70) | 27 | (67.5) |
| Duration of Treatment (DOT), | 5.55 | (0.6-16.00+) | 5.63 | (0.86-14.13+) | 5.60 | (0.6-16.00+) |
| median (range) | | | | | | |
| Duration of Response (DOR) in ORR | 7.53 | (1.9-13.16+) | 9.36 | (7.46-11.0) | 9.36 | (1.9-13.16+) |
| patients, median (range) | | | | | | |
| Duration of Clinical Benefit, | 9.36 | (1.9-14.13+) | 7.46 | (3.73-12.26+) | 7.46 | (1.9-14.13+) |
| median (range) | | | | | | |

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the appended claims define the scope of the invention and that methods and structures within the scope of these claims and their equivalents be covered thereby.

All publications, patents and patent applications cited in this application are herein incorporated by reference to the same extent as if each individual publication, patent, or patent application was specifically and individually indicated to be incorporated herein by reference.

1. A method of treating a solid tumor in a subject in need thereof comprising administering to the subject an effective amount of (i) a dual selective PI3K delta and gamma (PI3K δ/γ) inhibitor, (ii) a Salt-Inducible Kinase 3 (SIK3) inhibitor, or (iii) a combination thereof.

2. The method according to claim 1, wherein the dual selective PI3K delta and gamma (PI3K δ/γ) inhibitor is a compound of formula (A) 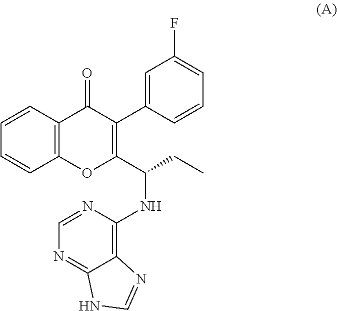 [SMILES: CC[C@H](Nc1ncnc2[nH]cnc12)c1oc2ccccc2c(=O)c1-c1cccc(F)c1]
or a pharmaceutically acceptable salt thereof.

3. The method according to claim 1, wherein the Salt-Inducible Kinase (SIK3) inhibitor is a compound of formula (M) 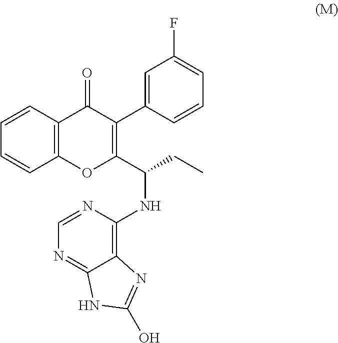 [SMILES: CC[C@H](Nc1ncnc2[nH]c(O)nc12)c1oc2ccccc2c(=O)c1-c1cccc(F)c1]
or a pharmaceutically acceptable salt thereof.

4. The method of claim 1, wherein the PI3K δ/γ inhibitor and/or the SIK3 inhibitor are administered in the form of a pharmaceutical composition.

5. The method according to claim 1, wherein the solid tumor is selected from carcinomas, sarcomas, hormonal solid cancers, and any combination of any of the foregoing.

6. The method according to claim 1, wherein the solid tumor is selected from pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancers, CNS cancers, brain tumors, bone cancer, soft tissue sarcoma, and any combination of any of the foregoing.

7. The method according to claim 1, wherein the solid tumor is selected from non-small cell lung cancer, small-cell lung cancer, colon cancer, CNS cancer, melanoma, ovarian cancer, renal cancer, prostate cancer, breast cancer, or any combination of any of the foregoing.

8. The method according to claim 1, wherein the solid tumor is breast cancer, ovarian cancer, colon cancer, pancreatic cancer, or any combination of any of the foregoing.

9. The method according to claim 1, wherein the solid tumor is breast cancer.

10. The method according to claim 9, wherein the breast cancer is selected from invasive breast cancer, non-invasive breast cancer, cancerous phyllodes tumors of the breast, locally advanced or metastatic breast cancer, hormone receptor (HR) positive and HER2 negative breast cancer, hormone receptor (HR) positive and HER2 positive breast cancer, hormone receptor (HR) negative and HER2 positive breast cancer, hormone receptor (HR) negative and HER2 negative breast cancer (triple negative breast cancer) and any combination of any of the foregoing.

11. The method according to claim 9, wherein the breast cancer is selected from invasive breast cancer, non-invasive breast cancer, cancerous phyllodes tumors of the breast and any combination of any of the foregoing.

12. The method according to claim 9, wherein the breast cancer is selected from locally advanced or metastatic breast cancer.

13. The method according to claim 9, wherein the breast cancer is selected from hormone receptor (HR) positive and HER2 negative breast cancer, hormone receptor (HR) positive and HER2 positive breast cancer, hormone receptor (HR) negative and HER2 positive breast cancer, and hormone receptor (HR) negative and HER2 negative breast cancer, any combination of any of the foregoing.

14. The method according to claim 9, wherein the breast cancer is hormone receptor (HR) positive and HER2 negative breast cancer.

15. The method according to claim 9, wherein the breast cancer is triple negative breast cancer (TNBC).

16. A method of treating a solid tumor in a subject in need thereof comprising administering to the subject an effective amount of (i) a dual selective PI3K delta and gamma (PI3K δ/γ) inhibitor, (ii) a Salt-Inducible Kinase 3 (SIK3) inhibitor, (iii) a combination of (i) and (ii), or (iv) a pharmaceutical composition comprising (i), (ii), or (iii), to the subject.

17. The method according to claim 1, wherein the PI3K δ/γ inhibitor is(S)-2-(1-(9H-purin-6-ylamino) propyl)-3-(3-fluorophenyl)-4H-chromen-4-one (a compound of formula (A)) or a pharmaceutically acceptable salt thereof, the SIK3 inhibitor is(S)-3-(3-fluorophenyl)-2-(1-((8-hydroxy-9H-purin-6-yl)amino) propyl)-4H-chromen-4-one (a compound of formula (M)) or a pharmaceutically acceptable salt thereof, and the subject is administered (i) the PI3K δ/γ inhibitor, (ii) the SIK3 inhibitor, (iii) a combination of (i) and (ii), or (iv) a pharmaceutical composition comprising (i), (ii), or (iii), by the oral, intravenous, intramuscular, or intraperitoneal route.

18. The method according to the claim 17, wherein (i) a compound of formula (A), or a pharmaceutically acceptable salt thereof, (ii) a compound of formula (M), or a pharmaceutically acceptable salt thereof, (iii) a combination of (i) and (ii), or (iv) a pharmaceutical composition comprising (i), (ii), or (iii), is administered to the subject by the oral route.

19. The method according to claim 17, wherein (i) a compound of formula (A), or a pharmaceutically acceptable salt thereof, (ii) a compound of formula (M), or a pharmaceutically acceptable salt thereof, (iii) a combination of (i) and (ii), or (iv) a pharmaceutical composition comprising (i), (ii), or (iii) is administered in solid form.

20. The method according to claim 1, wherein(S) -2-(1-(9H-purin-6-ylamino) propyl)-3-(3-fluorophenyl)-4H-chromen-4-one (a compound of formula (A)) or a pharmaceutically acceptable salt thereof is administered to the subject at a dose of about 25 mg to about 2400 mg, about 25 mg to 2 about 2000 mg, about 25 mg to about 1800 mg, about 25 mg to about 1600 mg, about 25 mg to about 1200 mg, about 25 mg to about 1000 mg, about 25 mg to about 800 mg, about 25 mg to about 600 mg, about 25 mg to about 400 mg, or about 25 mg to 200 mg.

21. The method according to claim 1, wherein(S) -2-(1-(9H-purin-6-ylamino) propyl)-3-(3-fluorophenyl)-4H-chromen-4-one (a compound of formula (A)) or a pharmaceutically acceptable salt thereof is administered to the subject at a dose of about 100 mg to about 2400 mg, about 100 mg to about 2000 mg, about 100 mg to about 1800 mg, about 100 mg to about 1600 mg, about 100 mg to about 1200 mg, about 100 mg to about 1000 mg, about 100 mg to about 800 mg, about 100 mg to about 600 mg, about 100 mg to about 400 mg, or about 100 mg to about 200 mg.

22. The method according to claim 1, wherein(S) -2-(1-(9H-purin-6-ylamino) propyl)-3-(3-fluorophenyl)-4H-chromen-4-one (a compound of formula (A)) or a pharmaceutically acceptable salt thereof is administered to the subject in a single dose or in multiple doses.

23. The method according to claim 1, wherein(S) -2-(1-(9H-purin-6-ylamino) propyl)-3-(3-fluorophenyl)-4H-chromen-4-one (a compound of formula (A)) or a pharmaceutically acceptable salt thereof is administered orally once or twice a day.

24. The method according to claim 1, wherein(S) -2-(1-(9H-purin-6-ylamino) propyl)-3-(3-fluorophenyl)-4H-chromen-4-one (a compound of formula (A)) or a pharmaceutically acceptable salt thereof is administered to the subject at a dose of about 200 to about 1200 mg twice daily, or about 400 to about 800 mg twice daily.

25. The method according to claim 1, wherein the method comprises administering to the subject an effective amount of the dual selective PI3K delta and gamma (PI3K δ/γ) inhibitor and a Salt-Inducible Kinase 3 (SIK3) inhibitor, and further comprises administering an anti-cancer treatment, an endocrine therapy, one or more cytostatic, cytotoxic, or anticancer agents, or any combination of any of the foregoing, to the subject.

26. The method according to claim 25, wherein the method comprises administering to the subject an effective amount of the dual selective PI3K delta and gamma (PI3K δ/γ) inhibitor and the Salt-Inducible Kinase 3 (SIK3) inhibitor, wherein the dual selective PI3K delta and gamma (PI3K δ/γ) inhibitor and the SIK3 inhibitor are administered simultaneously or sequentially.

27. The method according to claim 25, wherein the anti-cancer treatment is selected from chemotherapy, radiation therapy, biological therapy, bone marrow transplantation, stem cell transplant or any other anticancer therapy or with one or more cytostatic, cytotoxic or anticancer agents or targeted therapy either alone or in combination, such as, DNA interactive agents, such as fludarabine, cisplatin, Chlorambucil, Bendamustine or doxorubic in; alkylating agents, such as, cyclophosphamide; topoisomerase II inhibitors, such as etoposide; topoisomerase I inhibitors such as CPT-11 or topotecan; tubulin interacting agents, such as paclitaxel, docetaxel, or the epothilones (for example ixabepilone), either naturally occurring or synthetic; hormonal agents, such as tamoxifen; aromatase inhibitors, such as anastrozole, letrozole, exemestane, and testolactone; selective estrogen receptor degraders or downregulators (SERD), such as Fulvestrant, Giredestrant, Amcenestrant, AZD9833, Rintodestrant, LSZ102, LY3484356, Elacestrant, ZN-c5, D-0502 and SHR9549; thymidilate synthase inhibitors, such as 5-fluorouracil; and anti-metabolites, such as methotrexate; other tyrosine kinase inhibitors such as Iressa and OSI-774; angiogenesis inhibitors; EGF inhibitors; VEGF inhibitors; CDK inhibitors; SRC inhibitors; c-Kit inhibitors; Her1/2 inhibitors, Checkpoint kinase inhibitors, Salt induced kinase 3 (SIK3) inhibitors and monoclonal antibodies directed against growth factor receptors such as erbitux (EGF) and herceptin (Her2); CD20 monoclonal antibodies such as (HuMax; Intracel), AME-133v (LY2469298, Applied Molecular Evolution), and other anticancer agents such as Mundesine, Crizotinib, Romidepsin (Istodax), Belinostat, Pralatrexate (Folotyn), Gemcitabine, Alisertib (MLN8237), Dasatinib (Sprycel), E7777, Lenalidomide (Revlimid), Nelfinavir (VIracept), Panobinostat (LBH-589), Vorinostat (Zolinza), Everolimus (Afinitor), APO866,, Carfilzomib (Kyprolis), Mogamulizumab (KW-0761), and any combination of any of the foregoing.

28. The method according to claim 1, wherein the subject is refractory to chemotherapy treatment, or in relapse after treatment with chemotherapy.

29. The method according to claim 25, comprising administering a combination that includes one or more anticancer agents, cytostatic agents, cytotoxic agents, or any combination thereof.

30. The method according to claim 29, wherein one or more anticancer, cytostatic or cytotoxic agent are selected from PARP inhibitors, CDK inhibitors, MEK inhibitors, B-RAF inhibitors, m-TOR inhibitors, selective estrogen receptor degrader (SERD), HER-2 or EGFR inhibitors or dual inhibitors, PD1 inhibitors, RET inhibitors, or any combination of any of the foregoing.

31. The method according to claim 29, wherein the one or more anticancer agent is a PARP inhibitor.

32. The method according to claim 31, wherein the PARP inhibitor is olaparib or talzoparib.

33. The method according to claim 29, wherein the anticancer agent is a selective estrogen receptor degrader (SERD).

34. The method according to claim 33, wherein selective estrogen receptor degrader (SERD) is fulvestrant.

35. The method according to claim 25, wherein the one or more anticancer treatment is selected from chemotherapy, radiation therapy, biological therapy, bone marrow transplantation, stem cell transplantation, or any combination of any of the foregoing.

36. The method according to claim 35, wherein the anticancer treatment is chemotherapy with one or more chemotherapeutic agents.

37. The method according to claim 36, wherein the chemotherapeutic agent is selected from Taxol, Doxorubicin, Daunorubicin, Doxorubicin liposomal, Epirubicin, Idarubicin, Valrubicin, Carboplatin, Carmustine, Cisplatin, Cyclophosphamide, Dacarbazine, Ifosfamide, Lomustine, Melphalan, Temozolomide, Trabectedin, 5-fluorouracil, 6-mercaptopurine, Azacytidine, Capecitabine, Clofarabine, Cytarabine, Floxuridine, Fludarabine, Gemcitabine, Methotrexate, Pemetrexed, Pentostatin, Pralatrexate, Trifluridine, Tipiracil, and any combination of any of the foregoing.

38. The method according to claim 36, wherein the chemotherapeutic agent is Taxol, Doxorubicin, or a combination of any of the foregoing.

39. The method according to claim 1, wherein the subject is a human.
